(19) United States
(12) Patent Application Publication
(10) Pub. No.: US 2025/0217977 A1
(43) Pub. Date: Jul. 3, 2025

(54) REPRODUCIBLE IDENTIFICATION OF ACUTE-STAGE MILD TRAUMATIC BRAIN INJURY USING MACHINE LEARNING AND CONNECTOMICS

(71) Applicant: University of Southern California, Los Angeles, CA (US)

(72) Inventor: Andrei Irimia, Los Angeles, CA (US)

(73) Assignee: University of Southern California, Los Angeles, CA (US)

(21) Appl. No.: 19/003,820

(22) Filed: Dec. 27, 2024

Related U.S. Application Data
(60) Provisional application No. 63/616,501, filed on Dec. 29, 2023.

(51) Int. Cl. G06T 7/00; A61B 5/00; A61B 5/055; G06V 10/764

(52) U.S. Cl. CPC G06T 7/0012; A61B 5/0042; A61B 5/055; A61B 5/742; G06V 10/764; G06T 2207/10088; G06T 2207/30016; G06V 2201/031

(57) ABSTRACT

A system and method operates for diagnosis of mild traumatic brain injury (mTBI) to prevent sequelae and improve neurocognitive outcomes. The system may have a processor that receives, from a magnetic resonance imaging machine, data corresponding to magnetic resonance imaging images. By using a machine learning classifier of the processor, such as a Bayesian machine learning classifier, the processor identifies mild traumatic brain injury in a person through cortico-cortical connectome mapping from magnetic resonance imaging. The processor can also generate a human readable screen display indicative of the identified mild traumatic brain injury.

### CROSS-REFERENCE TO RELATED APPLICATIONS

This application is based upon and claims the benefit of and priority to U.S. Provisional Patent Application No. 63/616,501, filed on Dec. 29, 2023, entitled “Reproducible Identification of Acute-Stage Mild Traumatic Brain Injury Using Machine Learning and Connectomics,” the entire content of which is incorporated by reference herein.

### STATEMENT AS TO FEDERALLY SPONSORED RESEARCH

This invention was made with government support under grant number R01NS100973, awarded by the National Institute of Health (NIH). The government has certain rights in the invention.

### BACKGROUND

### 1. Field

The following disclosure relates to processing of medical imaging, and more specifically, to reproducible identification of acute-stage mild traumatic brain injury using machine learning and connectomics.

### 2. Description of Related Art

Accurate early diagnosis of mild traumatic brain injury (mTBI) is useful to prevent sequelae and improve neurocognitive outcomes. Early after head impact, mTBI diagnosis may be doubtful in persons whose neurological, neuroradiological, and/or neurocognitive examinations are equivocal. Moreover, conventional diagnostic tools may be limited in their ability to effectuate a diagnosis due to availability issues, co-extant and confounding problems, or the like. Thus, there remains a need to effectuate more accurate, more precise, and more readily available early diagnosis of traumatic brain injuries, and particularly, mild traumatic brain injuries.

### SUMMARY

A system for diagnosis of mild traumatic brain injury (mTBI) is provided. The system may be to prevent sequelae and improve neurocognitive outcomes. The system may include an imaging device. The imaging device may be a magnetic resonance imaging (MRI) machine configured to collect magnetic resonance images. The system may include a processor connected to the imaging device to receive the magnetic resonance images. The processor may include a Bayesian machine learning classifier. The processor may identify, using the Bayesian machine learning classifier, mild traumatic brain injury in a person through cortico-cortical connectome mapping from the magnetic resonance images. The system may include a screen display connected to the processor and configured to output a human readable depiction of the identifying.

In various embodiments, the Bayesian machine learning classifier is a software algorithm running on the processor, configured to identify acute mTBI in the absence of neuroradiological MRI findings on T1- or T2-weighted scans. In various embodiments, the Bayesian machine learning classifier is a software algorithm running on the processor, configured to discriminate or reveal TAI-related white matter (WM) connections or descriptors that are unusually sensitive to mTBI. In various embodiments, the Bayesian machine learning classifier is a software algorithm running on the processor, configured to perform a bilateral saliency analysis to alleviate the potential confounds of asymmetrical mTBI effects on neurocircuitry.

In various embodiments, the cortico-cortical connectome mapping includes analyzing, by the processor, multiple bilateral cortico-cortical connection pairs related to classification features. The bilateral cortico-cortical connection pairs link frontal lobes to limbic, temporal, parietal, and occipital structures, wherein the bilateral cortico-cortical connection pairs include 13 bilateral cortico-cortical connection pairs. The cortical structures linked by the 13 connections are displayed on a visual illustration of the cortex shown on the screen display. In various embodiments, the connectivity between the superior part of the precentral sulcus (located in the dorsolateral PFC) and the pericallosal sulcus (located in the limbic lobe) predicts and displays mTBI status on a display screen.

A method is provided for diagnosis of mild traumatic brain injury (mTBI). The method may be to prevent sequelae and improve neurocognitive outcomes. The method may include receiving by a processor and from a magnetic resonance imaging machine data corresponding to magnetic resonance imaging images. The method may include identifying, using a Bayesian machine learning classifier of the processor, mild traumatic brain injury in a person through cortico-cortical connectome mapping from magnetic resonance imaging. The method may include generating, by the processor, a human readable screen display indicative of the identified mild traumatic brain injury.

In various embodiments, the Bayesian machine learning classifier is a processor and/or software algorithm running on the processor, configured to identify acute mTBI in the absence of neuroradiological MRI findings on T1- or T2-weighted scans. In various embodiments, the Bayesian machine learning classifier is a processor and/or software algorithm running on the processor, configured to discriminate or reveal TAI-related white matter (WM) connections or descriptors that are unusually sensitive to mTBI. In various embodiments, the Bayesian machine learning classifier is a processor and/or software algorithm running on the processor, configured to perform a bilateral saliency analysis to alleviate the potential confounds of asymmetrical mTBI effects on neurocircuitry. In various embodiments, the cortico-cortical connectome mapping includes analyzing multiple bilateral cortico-cortical connection pairs related to classification features. In various embodiments, the bilateral cortico-cortical connection pairs link frontal lobes to limbic, temporal, parietal, and occipital structures. In various embodiments, the bilateral cortico-cortical connection pairs include 13 bilateral cortico-cortical connection pairs. In various embodiments, the cortical structures linked by the 13 connections are displayed on the cortex. In various embodiments, one connection links the frontal lobe to subcortical structures and to the parietal lobe, both ipsilaterally and contralaterally. In various embodiments, one connection between the superior frontal sulcus and the caudate nucleus spans a notable portion of frontal WM, where connectivity from superficial frontal areas travel to deeper subcortical regions. In various embodiments, these connections originate or terminate along the boundary between the somatomotor and somatosensory cortices, and their high sensitivity to mTBI reflects the cortical dynamics of post-traumatic pain syndromes. In various embodiments, the connectivity between the superior part of the precentral sulcus (located in the dorsolateral PFC) and the pericallosal sulcus (located in the limbic lobe) predicts and displays mTBI status on a display screen.

### BRIEF DESCRIPTION OF THE DRAWINGS

The subject matter of the present disclosure is particularly pointed out and distinctly claimed in the concluding portion of the specification. A more complete understanding of the present disclosure, however, may best be obtained by referring to the detailed description and claims when considered in connection with the following illustrative figures. In the following figures, like reference numbers refer to similar elements and steps throughout the figures.

FIG. 1 illustrates a system for diagnosis of mild traumatic brain injury, in accordance with various embodiments;

FIGS. 2A-D provides a diagrammatic illustration various connection pairs shown using a connectogram, in accordance with various embodiments;

FIGS. 3A-3B illustrate cortical structures linked by connections on the cortex, in accordance with various embodiments;

FIGS. 4A-4C provide images illustrate the links using streamlines, in accordance with various embodiments;

FIGS. 5-8 illustrate histograms showing connections among different regions of a brain, in accordance with various embodiments; and

FIG. 9 illustrates a method for diagnosis of mild traumatic brain injury, in accordance with various embodiments.

### DETAILED DESCRIPTION

A system, apparatus and/or method is provided for accurate early diagnosis of mild traumatic brain injury (mTBI) to prevent sequelae and improve neurocognitive outcomes, and is further described as set forth herein.

Accurate early diagnosis of mild traumatic brain injury (mTBI) is useful to prevent sequelae and improve neurocognitive outcomes. Early after head impact, mTBI diagnosis may be doubtful in persons whose neurological, neuroradiological, and/or neurocognitive examinations are equivocal. Such individuals can benefit from novel accurate assessments that complement clinical diagnostics. This disclosure introduces a Bayesian machine learning (ML) classifier to identify mTBI through cortico-cortical connectome mapping from magnetic resonance imaging (MRI) in persons with quasi-normal cognition and without neuroradiological findings. Classifier accuracy exceeds 99% in a discovery sample of 92 healthy controls (HCs) and 471 adult mTBI patients. This accuracy is replicated in an independent validation sample of 256 HCs and 126 mTBI patients. White matter (WM) connections enabling classification involve primarily prefrontal cortex, fronto-limbic and fronto-subcortical connections, and occipito-temporal structures in the ventral (“what”) visual stream. This and related connectivity form a “canary in the coal mine” network that is particularly vulnerable to mTBI. Because these connections are important in mediating cognitive control, memory, and attention, our findings explain the high frequency of cognitive disturbances after mTBI. This ML classifier can complement current diagnostics by providing independent information in clinical contexts where standard strategies for mTBI diagnosis stand to benefit from additional evidence.

In various regards, the disclosure is significant because mild traumatic brain injuries (mTBIs) can elude diagnosis when (A) magnetic resonance images lack neuroradiological findings and/or (B) subjective neurological/neurocognitive assessments do not enable confident evaluation of mTBI status. The disclosed connectome classifier enables near-ideal identification of acute mTBI in the absence of neuroradiological findings. Classifier accuracy exceeds 99% on discovery and independent validation samples, suggesting robust generalizability to new samples. The connectome features with highest predictive accuracy delineate a “canary in the coalmine” network of brain connections highly vulnerable to mTBI that explain the high incidence of behavioral disturbances and memory deficits after head injury. In conjunction with current diagnostics, this classifier provides an independent and objective source of diagnostic information in suspected mTBI cases where other evidence is ambiguous.

Traumatic brain injury (TBI) is a physical impact to the head associated with structural and functional disruptions to brain tissue. Patients with mild TBI (mTBI) may be at risk of accelerated brain aging and neurodegenerative conditions such as Alzheimer's disease. Acute diagnosis of mTBI is important to prevent sequelae and improve neurocognitive outcomes. However, mTBI is a heterogeneous condition that can be challenging to diagnose acutely due to (A) early neurocognitive symptoms that may be equivocal or insufficiently specific, (B) potential delay in the onset of such symptoms, or (C) frequent absence of focal lesions or injuries that are apparent on acute computed tomography (CT) or magnetic resonance imaging (MRI).

Conventionally, mTBI is diagnosed using standardized clinical assessments, which may include CT or MRI. The Glasgow Coma Scale (GCS), which classifies levels of consciousness, is frequently used to diagnose TBI and designate its severity. The GCS holds diagnostic power for moderate-to-severe TBI but is not sensitive to the symptoms and mental alterations—such as confusion, attention, and concentration problems—seen in mTBI. Cognitive tests assessing language, memory, and executive functioning can be used as evidence of mTBI. However, many neurocognitive batteries have low diagnostic power in distinguishing patients with mTBI from neurologically healthy controls (HCs). Variability in diagnosis also occurs due to potential subjectivity in the interpretation of clinical guidelines, leading to ˜50%-90% of mTBI cases going without formal diagnosis at hospital admission.

The standard diagnostic MRI protocols for suspected brain injuries include T₁- or T₂-weighted anatomic scans. Their primary utility is to provide radiologists with evidence of brain pathology and to differentiate between mTBI and moderate-to-severe TBI. However, the absence of identifiable lesions on T₁- or T₂-weighted MRIs does not rule out mTBI. For example, traumatic axonal injury (TAI) is not always detectable by such scans. In these and other cases, diffusion weighted imaging (DWI) can be used to assess mTBI impact on structural connectivity. TAI-related white matter (WM) disruption mapped using diffusion tensors estimated from DWI can be a major contributing factor to poor cognitive outcome. Tensors can be leveraged to quantify fractional anisotropy (FA), a surrogate measure of WM integrity defined as the directional coherence of water molecules in axonal bundles. Some studies suggest that, compared to HCs, FA is typically lower in mTBI patients within WM structures such as the corona radiata, cingulum, superior longitudinal and uncinate fasciculi, cortico-spinal tract, and corpus callosum. A shift from voxel-wise analysis towards whole brain connectomics may improve understanding of how mTBI affects macroscale neural networks. The integrity of WM connectivity, as conveyed by the mean FA of WM bundles between brain structures, is diminished in acute mTBI patients relative to HCs. Furthermore, FA-derived structural connectivity measures can distinguish the connectomes of mTBI patients from those of HCs. Thus, given mTBI patients' expected lack of neuroradiological findings on T₁- or T₂-weighted MRIs, the analysis of DWI-derived connectome features is a reasonable strategy to identify mTBI-related structural brain abnormalities.

This disclosure provides a Bayesian machine learning (ML) classifier to identify acute mTBI in the absence of neuroradiological MRI findings on T₁- or T₂-weighted scans. To generate classifier features, our workflow generates connectivity matrices specifying the mean FAs of WM connections between brain structures. Entries in these matrices become features for ML classifiers trained to detect mTBI in a discovery sample that includes both mTBI and HC participants, and in an independent validation sample. In both samples, the classifier identifies mTBI with accuracy above 99% in the absence of neuroradiological MRI findings, evidencing its ability to differentiate between mTBI and healthy brains in typical cases where diagnosis may be unclear. Classifier features with high discriminant ability reveal a “canary in the coalmine” network of WM connections that are unusually sensitive to mTBI and that are particularly useful for identifying this condition. Here, the canary in the coalmine is an allusion to caged canaries that miners would bring into tunnels. Toxic gas would kill canaries first, thereby providing a warning for miners to leave. In the present case, the canary in the coalmine is a set of descriptors (WM connectivity features) whose sensitivity to an adverse condition (mTBI) make them useful for identifying the condition early enough to provide useful treatments and psychoeducation.

Various practical implementations of a system for these innovations are provided and have been tested. For instance, a system for identifying acute brain injury, or stated differently, a system or method for diagnosis of mild traumatic brain injury (mTBI) to prevent sequelae and improve neurocognitive outcomes is provided. A discussion of this implementation follows below.

An implementation may be provided with various participants. In such instance, the definition of mTBI may be based on a) an acute GCS score above 12 at the time of the initial clinical examination; b) loss of consciousness shorter than 30 min; and c) post-traumatic amnesia lasting less than 24 hours. Study inclusion criteria included the availability of MRIs acquired up to 2 weeks (acute) post-injury. Participants who satisfied these criteria and who were both able and willing to provide written informed consent were invited to participate. Excluded were participants with a pre-traumatic history of clinical neurological disease, psychiatric disorder, or drug/alcohol abuse.

To train and validate classifiers, two samples were studied: a discovery (training) sample and a validation (testing) sample. The discovery sample comprised mTBI and HC participants from the Transforming Research and Clinical Knowledge in Traumatic Brain Injury (TRACK-TBI) Consortium, including 471 mTBI participants and 92 HCs (Table 1). The validation sample comprised 126 mTBI participants whose data were collected and available at the University of Southern California (henceforth labelled as the USC subsample, N=126) and 256 HCs from the Human Connectome Project (HCP subsample).

TABLE 1
Demographics of discovery and validation samples.

| | |
|---|---|
| discovery | validation |

| measure | HC | mTBI | HC | mTBI |
|---|---|---|---|---|
| sample size | 92 | 471 | 256 | 126 |
| males | 66 | 313 | 107 | 80 |
| females | 26 | 158 | 149 | 46 |
| age (years) | | | | |
| mean | 39.8 | 38.4 | 55.3 | 39.0 |
| standard deviation | 15.5 | 5.9 | 12.1 | 17.7 |

Cognitive assessments were summarized to calculate the average cognitive profiles of participants in each mTBI sample (discovery and validation), in each of which different tests were available. The discovery sample had three tests for both mTBI participants and HCs. The first is the Rey Auditory Verbal Learning Task, which evaluates verbal memory and learning ability through immediate and delayed recall of a list of 15 unrelated words. The second is the trail-making test, which quantifies visuomotor function, perceptual-scanning, and cognitive flexibility, with longer times indicating worse performance. The third test is the processing speed index of the Wechsler Adult Intelligence Scale, which includes two, timed tasks (the coding and symbol tasks, 120 seconds each). These tasks together index processing speed and visual motor coordination. The validation sample had the Brief Test of Adult Cognition by Telephone available for mTBI participants. The BTACT is a short phone-based assessment with six subtests to assess cognitive function implicated in pathology and aging: (1) episodic verbal memory—immediate recall (EVMI); (2) delayed recall (EVMD) of words on a 15-item list; (3) working memory span (WMS, assessed using a backward digit span task); (4) inductive reasoning (IR, assessed using a number series completion task); (5) processing speed (PS, measured using a backward counting task); and/or (6) verbal fluency (VF, assessed using a category fluency task). To situate mTBI participants in the discovery sample relative to the general (HC) population, mTBI cognitive scores were compared against those of 4,179 HCs from the Midlife in the United States (MIDUS) study.

First-order statistics (e.g., mean, standard deviation) of cognitive scores were calculated for mTBI participants and HCs in SPSS version 28. Independent-sample t-tests were used to compare mean differences between mTBI and HC in the discovery and validation samples separately (e.g., discovery mTBI vs. discovery HCs; validation mTBI vs. validation (MIDUS) HCs). Whenever Levene's test statistic was significant, unequal variances were assumed. Effect sizes of the differences between groups were assessed using Cohen's d, where effect sizes d<0.19 are negligible, 0.20<d<0.49 are small, 0.50<d<0.79 are medium, 0.80<d<1.20 are large, and d<1.20 are very large. Multiple comparisons were accounted for using a false discovery rate (FDR) correction.

The discussion will now turn to imaging in the discovery sample. For both mTBIs and HCs, MRIs were selected from the TRACK-TBI Consortium, which has standardized protocols for acquisition of imaging data (https://www.tracktbi.ucsf.edu/). T-weighted images were acquired in three dimensions with a multi-echo magnetization-prepared rapid gradient-echo sequence. DWIs were acquired with a multi-slice single-shot spin echo echo-planar pulse sequence (64 gradient directions; voxel size=2.7 mm×2.7 mm×2.7 mm; maximum b=1300 s/mm²; eight volumes with b=0 s/mm²). Scans were acquired on several approved scanners and underwent quality control to ensure compliance with all protocols necessary for inclusion in the TRACK-TBI Consortium.

The discussion will now turn to imaging in the validation sample. The MRIs of mTBI participants in the validation sample were acquired on a 3 T Prisma MAGNETOM Trio TIM scanner (20-channel head coil, Siemens Corporation, Erlangen, Germany). T₁-weighted images were acquired in three dimensions using a magnetization-prepared rapid gradient-echo sequence (voxel size=1.0 mm×1.0 mm×1.0 mm; repetition time (T_{R})=1.95 s; echo time (T_{E})=2.98 s; inversion time (T₁)=0.9 s). DWIs were acquired axially in 64 gradient directions (voxel size=2.73 mm×2.73 mm×2.7 mm; T_{R}=8.3 s; T_{E}=72 ms; maximum b=1300 s/mm²; one volume with b=0 s/mm²). For HCs, MRIs were selected from the HCP-Aging repository (https://www.humanconnectome.org/study/hcp-lifespan-aging), acquired on a 3 T PRISMA scanner (32-channel head coil, Siemens Corporation, Erlangen, Germany). T₁-weighted MRIs were acquired in three dimensions with a multi-echo magnetization-prepared rapid gradient-echo sequence (voxel size=0.8 mm×0.8 mm×0.8 mm; T_{R}=2.5 s; T_{E}=2.22 ms; T_{I}=1 s). DWIs were acquired axially in 98 gradient directions (voxel size=1.5 mm×1.5 mm×1.5 mm; T_{R}=3.23 s; T_{E}=89.20 ms; maximum b=1300 s/mm²; seven volumes with b=0 s/mm²).

The discussion will now turn to image processing. T₁-weighted MRIs were pre-processed and segmented automatically using Freesurfer 6.0 (http://surfer.nmr.mgh.harvard.edu/), with default parameters and a standard protocol described elsewhere. Non-cortical structures were stripped using a hybrid-watershed deformation process, image intensities were normalized, and volumes were registered into Talairach space. Segmentation followed the Destrieux parcellation scheme, which contains 165 structures: 74 cortical and 8 subcortical in each hemisphere, as well as one brain stem structure.

DWIs were processed using 3DSlicer 4.11 (https://www.slicer.org/) and DTIPrep extension 0.1.1 (https://www.nitrc.org/projects/dtiprep/). Skull-stripped DWIs and b₀ images were registered to T₁-weighted volumes using the BRAINSFit module of 3DSlicer. Any DWI volume with poor registration to the corresponding T₁-weighted volume was corrected with user supervision using a transformation matrix estimated from the registration between the b₀ volume and the original T₁-weighted volume. Unscented Kalman filter (UKF) two-tensor tractography was performed in 3DSlicer using whole-brain seeding, with default parameters described elsewhere and one seed per voxel. UKF tractography is a deterministic tractography approach that fits two tensors at each step. The UKF algorithm utilizes previous tracking positions to direct model estimation and to improve tracking. FA was calculated from UKF-derived tensors at each voxel. Tractograms were co-registered onto anatomical segmentations of the brain. Streamlines shorter than 1.5 cm were discarded due to their higher likelihood to be spurious. The quality of participants' imaging data was verified by a statistical quality control process described below.

Regarding a connectivity calculation that was utilized, connectivity matrices specifying the mean FAs of connections between all pairs of brain structures were constructed using purpose-built software. Let M and N be the number of brain structures and subjects, respectively. The connectivity matrix C has scalar entries cᵢⱼₖ, where i=1, . . . , M, j=1, . . . , M, and k=1, . . . , N. Each connectivity matrix vector cᵢⱼ of length N describes, for subjects 1, . . . , N, the mean FA of tractography streamlines between i and j. For each pair i and j, the implementation computed the fraction of subjects for whom cᵢⱼₖ≠0, i.e., who had a WM connection between i and j. For each cᵢⱼ, the interquartile range (IQR) of mean FAs was calculated, as were the lower, middle, and upper quartiles (Q₁, Q₂ and Q₃, respectively). The effort attempted to exclude participants with cᵢⱼₖ<Q₁−1.5×IQR or cᵢⱼₖ>Q₃+1.5×IQR but none satisfied either of these inequalities. To reduce spurious connectivity due to noise and tractography artifacts, the implementation removed outlier participant information from C. Specifically, connections present in fewer than 80% of participants in each cohort (mTBI-discovery, HC-discovery, mTBI-validation, HC-validation) were removed because such connections were more likely to be artifactual and less likely to be sampled adequately. Nonzero entries left in each vector cᵢⱼ were normalized by dividing them by their maximum value across subjects. To account for demographic variable effects on cᵢⱼ, linear regressions were implemented to partial out the statistical effects of age, sex, and both their first- and second-order interactions (independent variables) on FA (dependent variable).

The discussion now continues with machine learning classification and interpretability. To classify participants as mTBI or HC, the implementation trained 25 standard supervised ML classifiers available in MATLAB's classification learner, using five-fold cross-validation to alleviate overfitting. For each classifier, in addition to predictive accuracy (i.e., the percentage of participants whose diagnostic status was identified correctly), the implementation calculated sensitivity (e.g., true positive rate, TPR), specificity (e.g., true negative rate, TNR), precision (e.g., positive predictive value, PPV), and negative predictive value (NPV). From the set of trained classifiers, the implementation chose an optimal one for further analysis based upon predictive accuracy and overall suitability to the computational problem. This classifier was applied to the validation sample, where classification measures were calculated.

To rank connections according to their classification utility (saliency), the implementation trained distinct instantiations of the optimal model which only included a pair of bilateral features comprising a single connection and its contralateral homolog. For example, to quantify the classification saliency of hippocampo-amygdalar connectivity, the implementation included both (A) the connection between the right hippocampus and the right amygdala and (B) the connection between the left hippocampus and the left amygdala. The implementation opted for this type of bilateral saliency analysis to alleviate the potential confounds of asymmetric mTBI effects on neurocircuitry. Inclusion of bilateral feature pairs contributes to reducing such confounds because it allows study of the bilateral saliency of a connection to the classifier rather than the unilateral asymmetry of injury effects on that connection.

To provide model interpretability, the implementation computed classifier sensitivity and specificity when it was trained using each bilateral pair (left and right homologs) of features. Both measures were evaluated as indicators of classifier performance to ensure that our findings were sensible. The implementation sought to confirm that connections deemed most salient for classification exhibited mean FAs that differed significantly between classes (mTBI vs. HC subjects). This step helped to ensure that high classification accuracy was not due to artifactual or erroneous mean FA differences between classes that might confound findings. For confirmation, the empirical probability distribution functions (PDFs) of mean FA were calculated for each discovery sample class and for each connection. The null hypothesis of no group difference in mean FA PDFs was tested using the Kolmogorov-Smirnov (KS) goodness of fit test. Its test statistic quantified whether the mean FA PDF of the most useful connections differed between classes. The implementation hypothesized that this difference was significant for connections with highest individual predictive accuracy and nonsignificant for those with lowest individual accuracy.

The results of the implementation are provided below, first with regard to cognitive and imaging profiles. In various embodiments, and consistent with mTBI diagnosis, all participants lack neuroradiological findings. Within the discovery sample, mTBI participants recalled significantly fewer words (around 9 on average, compared to around 10 by HCs) from the RAVLT immediately after learning. However, Cohen's d is small (d=−0.301; Table 2). There are no other differences between mTBI and HCs in the discovery sample after FDR correction. More than 93% of mTBI participants have cognitive scores within the HC normal range (e.g., z>−1.96). In other words, the vast majority of mTBI participants' cognitive scores are not meaningfully distinguishable from HCs'. In the validation sample, mTBI participants' average BTACT scores are significantly lower than HCs' (Table 2). For instance, on both immediate and delayed recall tasks, the average mTBI participant remembered half the number of words that HCs did and could retain one fewer digit in the working memory task. mTBI participants were able to name, on average, 13 animals/fruit in 60 seconds, while HCs could name almost 19. A medium-sized effect (d=−0.481) of mTBI diagnosis on IR scores was observed, where the negative sign indicates that mTBI participants' mean scores are lower than HCs'. All other effect sizes are large (d=−0.697 to d=−0.891) except for EVMI (where the effect is very large, d=−1.242). Despite significantly lower average mTBI scores relative to HCs, individual scores are largely indistinguishable, in a statistical sense, from those of HCs (Table 2). Specifically, no mTBI participant exceeds the two-sigma cut-off (z<−1.96) for ‘poor’ EVMD, WMS, or IR scores. Only for a small minority of mTBI participants are PS and VF z-scores significantly lower than those of HCs (15% and 18%, respectively), and fewer than half (39%) have EVMI z-scores below the cut-off.

Table 2, reproduced herein, illustrates means, standard deviations, and independent samples t-tests comparing mTBI to HC cognitive scores on available tests. Cognitive scores in the mTBI validation subsample were compared to those of HCs in MIDUS. There are several important points to consider regarding an evaluation of Table 2. The statistic of Levene's test for equality of variances between mTBI and HC cognitive scores was significant, so equal variances were not assumed. Percentage of mTBI patients whose cognitive score was within the normal range of HCs (e.g., whose z-scores were above the left-tailed two-sigma cutoff of −1.96 for the listed task). Abbreviations: dx=diagnosis (mTBI or HC), N=sample size, μ=mean, σ=standard deviation, t=independent samples t-value, df=degrees of freedom, p=two-tailed significance value, μ_{Δ}=mean difference, σ_{Δ}=standard error of the difference, d=Cohen's d. RAVLT IR=Rey auditory verbal learning task immediate recall, RAVLT DR=RAVLT delayed recall, TMT A=trail-making test trial A time, TMT B=TMT trial B time, WAIS PS=Weschler adult intelligence scale processing speed (standardized).

TABLE 2
Comparing mTBI to HC cognitive scores.

| | | | | | | | | | | | |
|---|---|---|---|---|---|---|---|---|---|---|---|
| test | dx | N | μ | σ | t | df | p | μ_{Δ} | σ_{Δ} | d | % ^{a} |

discovery sample

| | | | | | | | | | | | |
|---|---|---|---|---|---|---|---|---|---|---|---|
| RAVLT | mTBI | 457 | 9.53 | 3.17 | −2.585 | 543 | 0.010 | −0.95 | 0.37 | −0.301 | 93 |
| IR | | | | | | | | | | | |
| | HC | 88 | 10.48 | 3.06 | | | | | | | |
| RAVLT | mTBI | 456 | 9.22 | 3.29 | −1.826 | 542 | 0.068 | −0.70 | 0.38 | −0.213 | 96 |
| DR | | | | | | | | | | | |
| | HC | 88 | 9.92 | 3.33 | | | | | | | |
| TMT A | mTBI | 456 | 30.39 | 15.07 | 2.298 | 540 | 0.022 | 3.94 | 1.72 | 0.270 | 100 |
| | HC | 86 | 26.45 | 11.75 | | | | | | | |
| TMT B | mTBI | 453 | 74.38 | 40.29 | 1.227 | 537 | 0.220 | 5.75 | 4.68 | 0.144 | 100 |
| | HC | 86 | 68.63 | 37.23 | | | | | | | |
| WAIS PS | mTBI | 454 | 99.88 | 16.15 | −0.136 | 538 | 0.892 | −0.26 | 1.89 | −0.016 | 97 |
| | HC | 86 | 100.14 | 15.37 | | | | | | | |

validation sample

| | | | | | | | | | | | |
|---|---|---|---|---|---|---|---|---|---|---|---|
| EVMI | mTBI | 110 | 3.820 | 2.72 | −10.955 | 113 | <.001 | −2.862 | 0.26 | −1.242 | 61 |
| | HC | 4493 | 6.680 | 2.29 | | | | | | | |
| EVMD | mTBI | 110 | 2.230 | 2.34 | −8.428 | 4384 | <.001 | −2.137 | 0.25 | −0.814 | 100 |
| | HC | 4276 | 4.360 | 2.63 | | | | | | | |
| WMS | mTBI | 110 | 3.780 | 1.58 | −8.028 | 4604 | <.001 | −1.188 | 0.15 | −0.775 | 100 |
| | HC | 4496 | 4.960 | 1.53 | | | | | | | |
| IR | mTBI | 110 | 1.450 | 1.30 | −6.166 | 117 | <.001 | −0.780 | 0.13 | −0.481 | 100 |
| | HC | 4512 | 2.230 | 1.63 | | | | | | | |
| PS | mTBI | 110 | 28.451 | 13.34 | −6.399 | 113 | <.001 | −8.219 | 1.28 | −0.697 | 85 |
| | HC | 4479 | 36.670 | 11.75 | | | | | | | |
| VF | mTBI | 110 | 13.066 | 7.03 | −8.152 | 113 | <.001 | −5.516 | 0.68 | −0.891 | 82 |
| | HC | 4498 | 18.582 | 6.17 | | | | | | | |

Regarding ML classification, no significant relationship of age, sex, and/or their interaction on mean FA was found (p>0.05, corrected). Of 25 supervised probabilistic classifiers tested, 17 could be trained on the discovery sample. Two classifiers achieved 100% predictive accuracy in the discovery sample (Table 3), while 8 classifiers—the support vector machine and discriminant models, both linear and quadratic—were discarded from ulterior analysis because they failed to converge. The implementation also tested k-nearest neighbor classifiers (fine, medium, coarse, cosine, cubic, weighted, and subspace ensemble varieties), logistic regression, boosted trees ensemble, and subspace discriminant ensemble classifiers. These yielded 83.7% accuracy as they incorrectly classified all HCs as mTBI participants. Two naïve (Gaussian and kernel) Bayesian classifiers achieved accuracy above 99% on the discovery sample. The Gaussian classifier assumes that features are normally distributed, but the kernel classifier makes no such assumptions, being more flexible in its ability to model nonlinear class boundaries. Compared to Bayesian classifiers, decision trees (both standard and ensemble) perform similarly well and are more parsimonious. However, they are also more vulnerable to overfitting and more reliant upon features with low predictive power. Decision trees are less suited to the discussed setting due to the relatively unbalanced nature of the samples (discovery sample: 471 mTBIs and 92 HCs; validation sample: 126 mTBIs and 256 HCs). Both naïve Bayesian classifiers trained here are scalable and robust to overfitting, but Gaussian iteration is better for features comprising continuous variables such as FA. By contrast, kernel classifiers are typically more suitable for categorical or discrete data. For these reasons, the Gaussian naïve Bayesian classifier was selected for further analysis. This classifier can identify mTBI participants in the validation sample with accuracy above 99%.

Table 3 illustrates classification error rates on the discovery sample for a selected subset of well-performing approaches, expressed in percentages. Table 3 has various abbreviations, including: TPR=true positive rate (i.e., sensitivity), TNR=true negative rate (i.e., specificity), PPV=positive predictive value (i.e., precision), NPV=negative predictive value (i.e., miss rate).

TABLE 3
Classification Error Rates on the Discovery Sample

| | | | | | |
|---|---|---|---|---|---|
| classifier | accuracy | TPR | TNR | PPV | NPV |

naïve Bayesian classifiers

| | | | | | |
|---|---|---|---|---|---|
| kernel | 100.00 | 100.00 | 100.00 | 100.00 | 100.00 |
| gaussian | 100.00 | 100.00 | 100.00 | 100.00 | 100.00 |

decision trees

| | | | | | |
|---|---|---|---|---|---|
| fine | 99.50 | 100.00 | 96.74 | 99.37 | 100.00 |
| medium | 99.50 | 100.00 | 96.74 | 99.37 | 100.00 |
| coarse | 99.50 | 100.00 | 96.74 | 99.37 | 100.00 |

ensemble methods

| | | | | | |
|---|---|---|---|---|---|
| bagged | 99.50 | 100.00 | 96.74 | 99.37 | 100.00 |
| rus boosted | 95.60 | 99.15 | 77.17 | 95.70 | 94.67 |

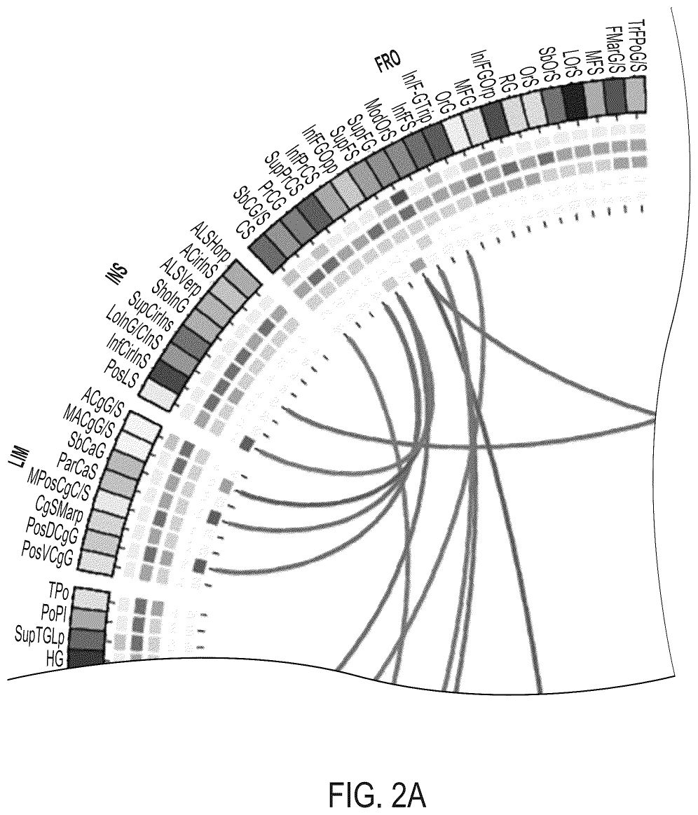

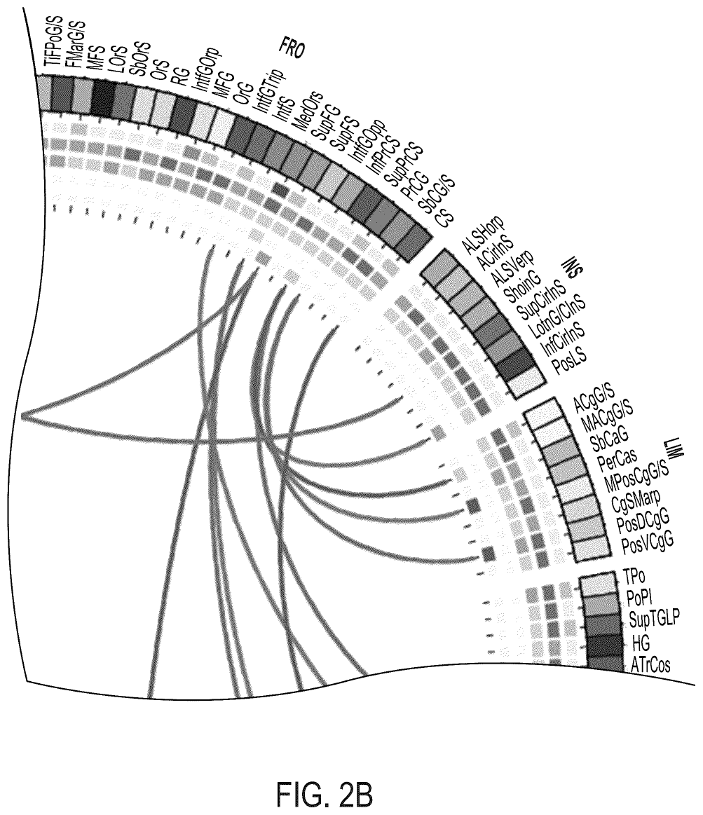

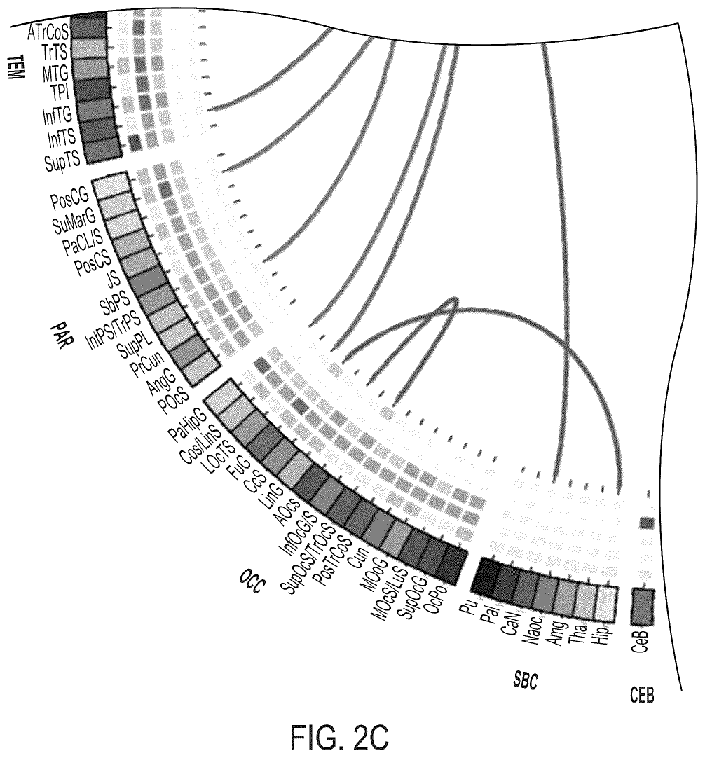

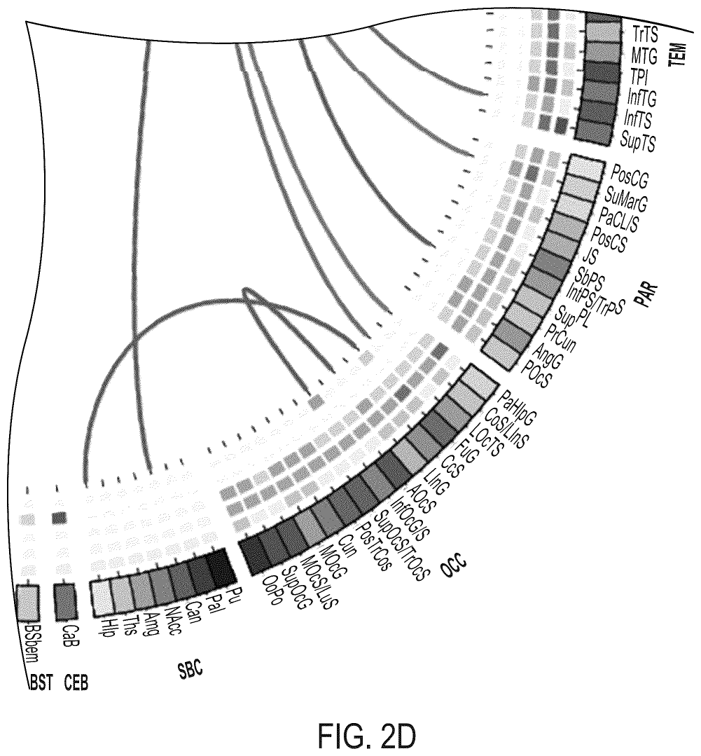

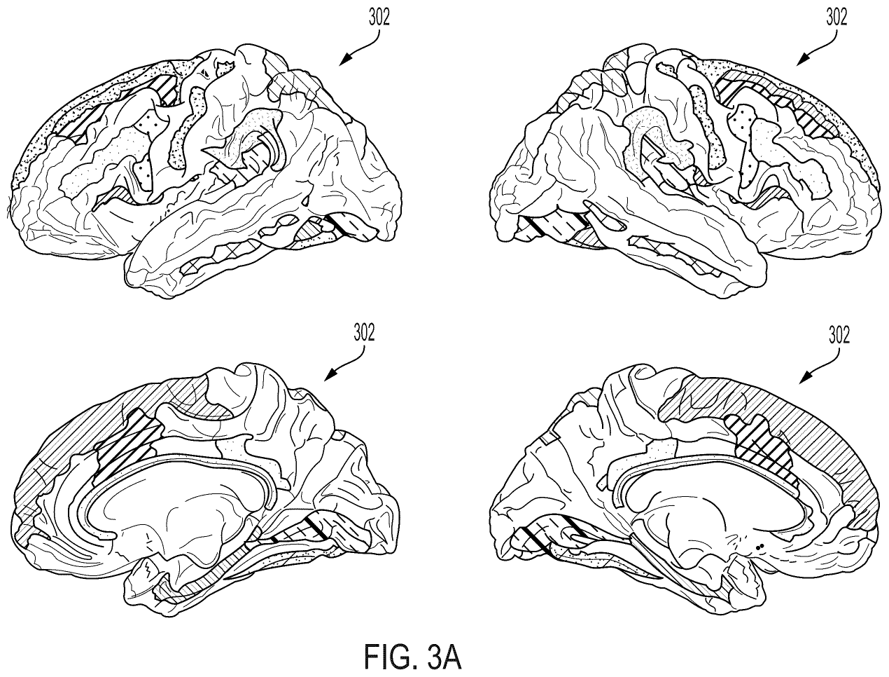

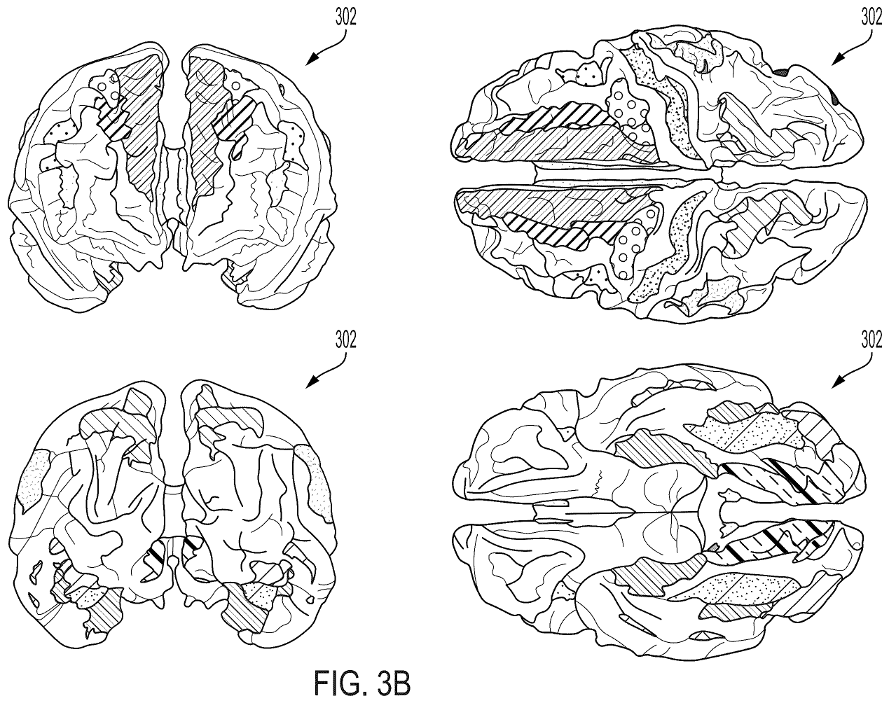

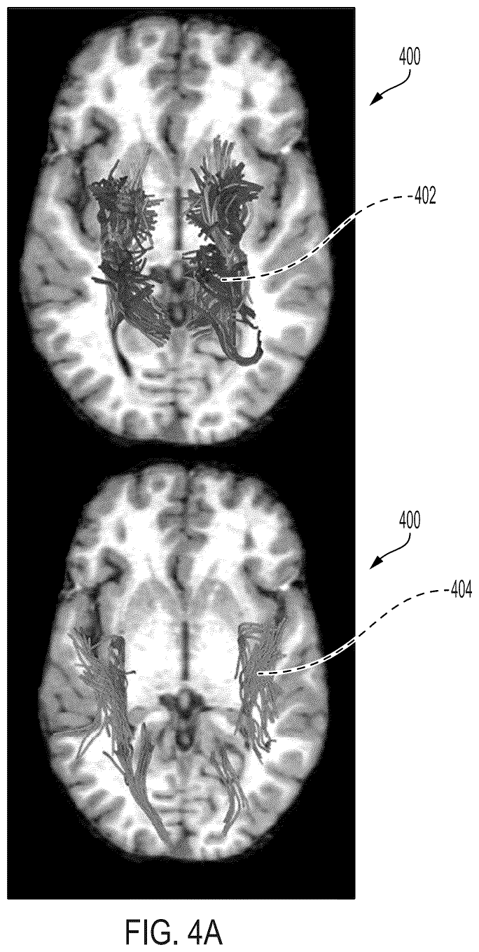

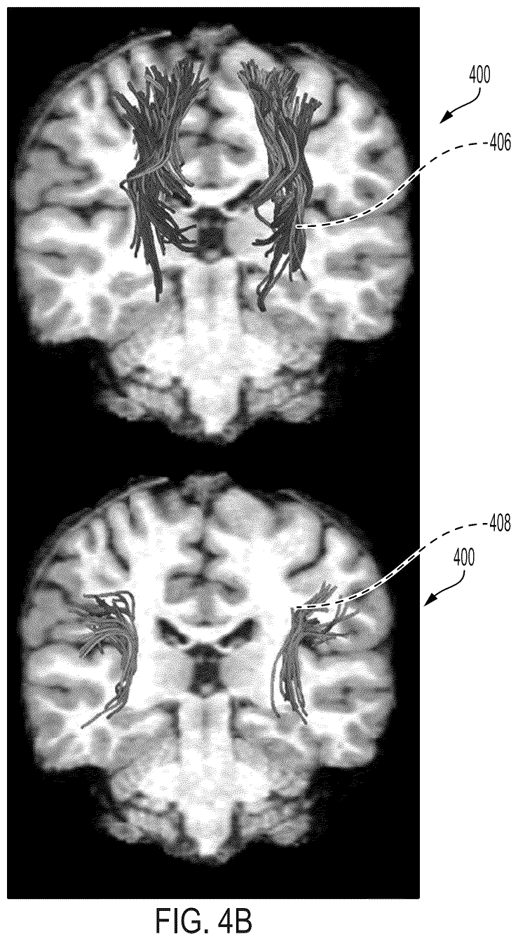

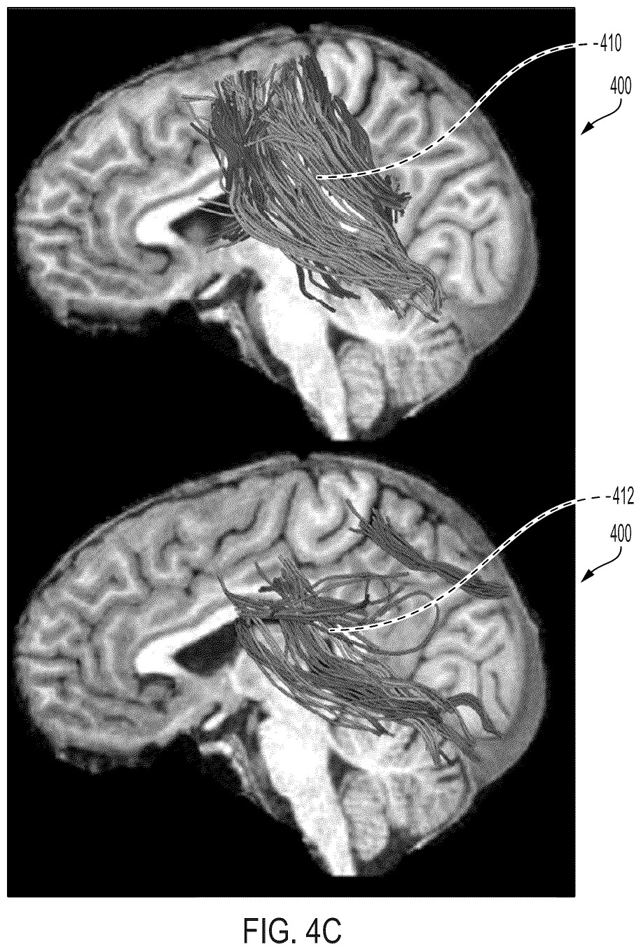

The analysis singles out brain connections that are particularly sensitive and specific to mTBI (Table 4). Notably, thirteen bilateral cortico-cortical connection pairs are related to (e.g., from or forming) classification features predicting diagnostic status with accuracy higher than 99% in both discovery and validation samples. These connection pairs link frontal lobes to limbic, temporal, parietal, and occipital structures. FIGS. 2A-D provides a diagrammatic illustration 200 of these connection pairs shown using a connectogram. FIGS. 3A-3B illustrate cortical structures linked by these 13 connections on the cortex 302. FIGS. 4A-4C provide images 400 illustrate the links using streamlines 402, 404, 406, 408, 410, and 412. One connection pair with high predictive accuracy links the occipital lobes to the subcallosal aspects of the ipsilateral limbic lobes (see FIGS. 2A-D). The two connections pairs with highest predictive accuracy link (A) the fusiform gyrs to the ipsilateralhippocampus and (B) pericallosal to precentral sulci, ipsilaterally (see Table 4). To further contextualize, the connections most representative of mTBI, those that predict diagnostic status with accuracy above 95% in both discovery and validation samples may be visualized. A notable proportion of such connections link the frontal lobe to subcortical structures and to the parietal lobe, both ipsilaterally and contralaterally. Table 4 illustrates Bayesian classification accuracies of single features conveying the mean FA of cortico-cortical connectivity between two regions.

TABLE 4
Bayesian Classification Accuracies

| | | accuracy | | sensitivity | |
|---|---|---|---|---|---|
| region 1 | region 2 | discovery | validation | validation | validation |
| pericallosal sulcus | precentral | 100.00% | 100.00% | 100.00% | 100.00% |
| | sulcus, superior | | | | |
| | part | | | | |
| fusiform gyrus | hippocampus | 99.64% | 100.00% | 100.00% | 100.00% |
| inferior occipital gyrus | lingual gyrus | 99.47% | 100.00% | 100.00% | 100.00% |
| and sulcus | | | | | |
| superior frontal gyrus | parahippocampal | 99.47% | 100.00% | 100.00% | 100.00% |
| | gyrus | | | | |
| superior frontal sulcus | circular insular | 99.47% | 100.00% | 100.00% | 100.00% |
| | sulcus, superior | | | | |
| | part | | | | |
| posterior ramus of the | precentral | 99.64% | 99.74% | 99.21% | 100.00% |
| lateral sulcus | sulcus, inferior | | | | |
| | part | | | | |
| superior parietal lobule | central sulcus | 99.30% | 100.00% | 100.00% | 100.00% |
| cingulate gyrus and | precentral | 99.82% | 99.48% | 98.41% | 100.00% |
| sulcus, middle- | sulcus, inferior | | | | |
| anterior part | part | | | | |
| superior frontal gyrus | lateral occipito- | 99.30% | 100.00% | 100.00% | 100.00% |
| | sulcus | | | | |
| precentral sulcus, | inferior temporal | 99.12% | 100.00% | 100.00% | 100.00% |
| inferior part | sulcus | | | | |
| supramarginal gyrus | inferior frontal | 99.11% | 100.00% | 100.00% | 100.00% |
| | sulcus | | | | |
| cingulate gyrus, | precentral | 99.30% | 99.74% | 99.21% | 100.00% |
| posterior-dorsal part | sulcus, superior | | | | |
| | part | | | | |
| superior frontal sulcus | caudate nucleus | 99.64% | 99.21% | 100.00% | 98.83% |

Referring in detail to FIGS. 2A-D, an illustration 200 depicts connectograms of connectivity features that best facilitate mTBI identification as part of a “canary in the coalmine” network of brain connections, e.g., connections with above 99% accuracy in both discovery and validation cohorts. The outermost ring encodes brain structures arranged by lobe (fr—frontal; ins—insula; lim—limbic; tem—temporal; par—parietal; occ—occipital; nc—non-cortical; bs—brain stem; CeB—cerebellum). Lobes and structures are ordered from anterior to posterior according to their center-of-mass locations within the right-anterior-superior (RAS) brain coordinate system. Each lobe has a unique dominant shading scheme with distinct shading for each constituent structure. Connectogram links encode the mean FA of streamlines forming the respective connection between any two structures. Some shaded links denote tractography-resolved pathways in the lower tercile of the mean FA distribution (lowest mean FAs), some links are pathways with mean FAs in the middle tercile, and other links are in the top tercile (highest mean FA).

Referring in detail to FIGS. 3A-B, brain structures are linked to other parts of the brain by connections with optimal discriminative ability to identify mTBI. Brain structures involved in connections that are optimal for mTBI classification are shown with views including left lateral, right lateral, frontal, and superior, medial right, medial left, posterior, and inferior.

Referring in detail to FIGS. 4A-C, streamline representations of WM connections whose mean FAs have the highest classification accuracy to identify mTBI. Structures connected by these streamlines are shown in FIGS. 3A-B, and the connections are visualized in the connectogram in FIGS. 2A-D. Streamlines have different mean streamline orientation (some streamline trajectories primarily span the sagittal plane, i.e., they proceed from the anterior to the posterior part of the brain; other streamlines are along the coronal plane, i.e., inferior to superior; still other streamlines are along the transverse plane, i.e., left to right). Streamline bundles with orientations largely along the coronal plane linking the posterior-dorsal part of the cingulate gyrus to the precentral sulcus, the superior part of the superior frontal gyrus to the parahippocampal gyrus, the superior frontal gyrus to the lateral occipito-temporal sulcus, the superior frontal sulcus to the caudate nucleus, and the pericallosal sulcus to the superior part of the precentral sulcus are shown. Also, streamline bundles with orientations largely along the sagittal plane linking the fusiform gyrus to the hippocampus, the supramarginal gyrus to the inferior frontal sulcus, the superior parietal lobule to the central sulcus, the inferior occipital gyrus and sulcus to the lingual gyrus, the posterior ramus of the lateral sulcus to the inferior part of the precentral sulcus, and the inferior part of the precentral sulcus to the inferior temporal sulcus are shown.

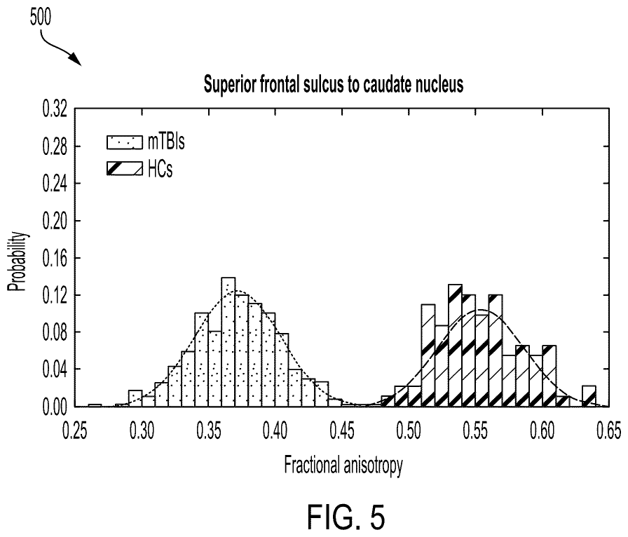

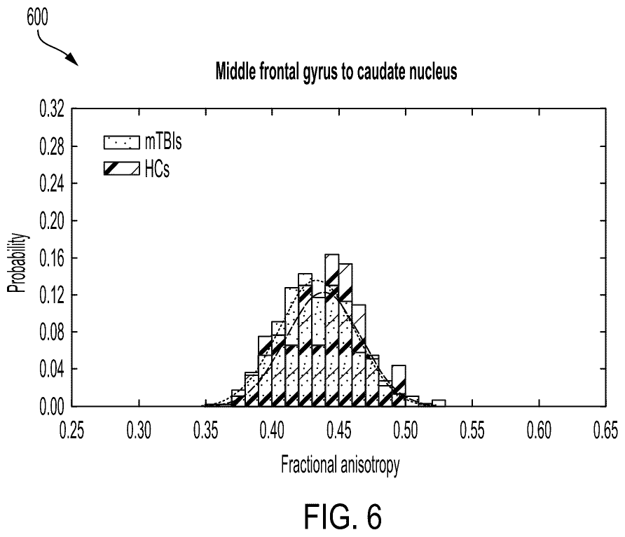

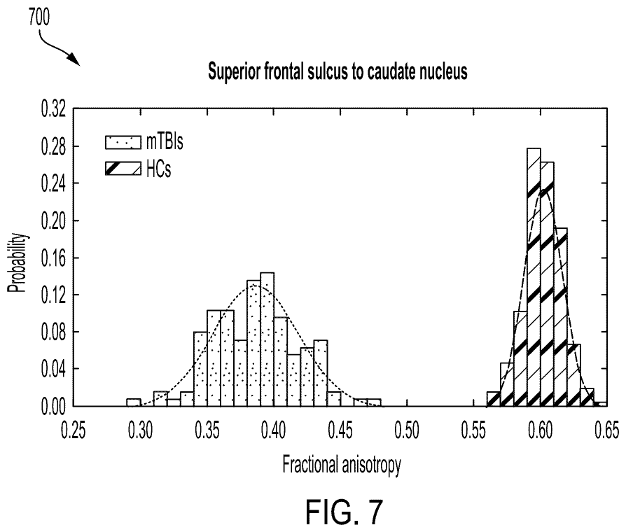

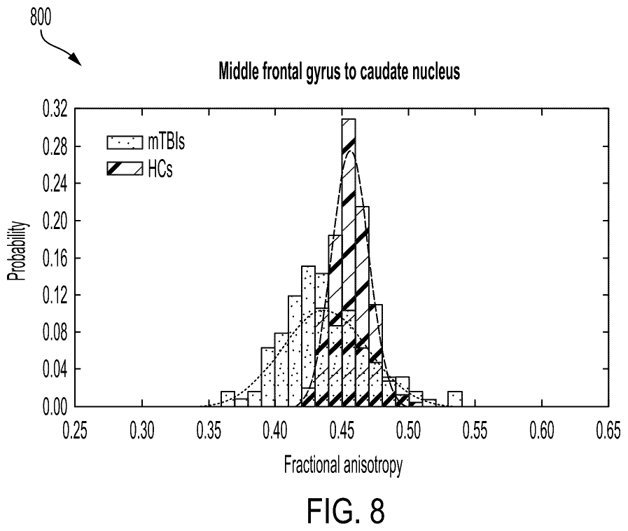

Finally, referring in detail to FIGS. 5-8, comparison of mean FA for representative connections is provided. FIGS. 5-6 illustrate aspects of a discovery sample. FIGS. 7-8 illustrate aspects of a validation sample. In FIGS. 5-8, histograms of mean FA are depicted for two representative connections: one whose individual classification accuracy is near-ideal (superior frontal sulcus to the caudate nucleus, accuracy >99%, left column) and one whose accuracy is poor (middle frontal gyrus to the caudate nucleus, accuracy=9.95%, right column) (see also Table 4). Mean FA histograms were compared across groups for each bilateral connection pair. For instance, FIG. 5 illustrates a histogram 500 showing connection from superior frontal sulcus to caudate nucleus. For instance, FIG. 6 illustrates a histogram 600 showing connection from middle frontal gyrus to caudate nucleus. FIG. 7 illustrates a histogram 700 showing connection from superior frontal sulcus to caudate nucleus. FIG. 8 illustrates a histogram 800 showing connection from middle frontal gyrus to caudate nucleus. With reference to FIGS. 5-8, for the 13 connection pairs with highest predictive accuracy, a two-sample KS test rejected the null hypothesis that the mean FAs of mTBI and HC subjects were from the same continuous distribution (p<0.05). This confirms that brain features enabling the classifier's high discriminant ability reflect genuine diffusion properties (mean FA) of connections on which classification relies. By contrast, for the 13 connections with lowest prediction accuracy, the null hypothesis of the KS test was not rejected (p>0.05).

The analysis identified 1,265 connections confidently, and their sensitivity and specificity were tallied. The sum of TPR and TNR is above 1.65 in both discovery and validation cohorts for 50 connections (3.95% of all connections). Other studies have suggested that tests with accuracies above 90% are “excellent” for diagnosis, whereas accuracies between 75% to 90% have “good” diagnostic value. By this measure, 279 connections (22.06%) have excellent accuracies in both discovery and validation cohorts, while 181 (14.31%) connections have good accuracies in both discovery and validation cohorts. A total of 805 connections (63.63%) has discovery or validation accuracies below 75%, suggesting that these features would be diagnostically suitable neither on their own nor as part of a diagnostic classifier or panel. Together, these findings confirm the appraisal of the Bayesian classification herein and lend credibility to the premise that the analysis is based on both direct quantitative measures of brain water diffusion and sound inferences.

The following discussion will address various topics related to the analysis, beginning with a discussion of various significances of various aspects, and continuing with other topics. Often, neuropsychological assessment for mTBI is made only weeks to months after the injury. This can be due to the perceived absence or mildness of neuropsychological symptoms in the acute phase of injury. In the post-acute phase of injury, a considerable proportion of mTBI victims experience persistent cognitive deficits such as memory difficulties, distractibility and impulsivity, and impaired information processing. Patients with acute mTBI who are misdiagnosed do not receive necessary treatments and psychoeducation, and thus are at greater risk of complications during recovery. Because the classifier was trained on the MRIs of acute mTBI patients without neuroradiological findings, this approach is particularly appealing for clinical cases where imaging examinations do not corroborate a diagnosis of mTBI. The lack of MRI findings frequently reflects a lack of CT findings but does not guarantee it. Thus, the classifier is less relevant for acute mTBI patients with CT findings but without MRI findings.

The classifier was tested on participants with mTBI who lack neuroradiological findings, but some of whom exhibited cognitive symptoms that allowed unambiguous mTBI diagnosis. Thus, one hypothetical concern is that the classifier may only be useful for identifying mTBI in the presence of such symptoms. However, the mTBI discovery sample includes participants who, on average, exhibit deficits in EVMI but are otherwise not impaired significantly compared to HCs. mTBI participants in the validation sample exhibit, on average, deficits in working and episodic verbal memory, slowed processing, impaired inductive reasoning, and limited verbal fluency. In both samples, on average, mTBI participants' scores are lower than HCs'. Nevertheless, individual mTBI participants' cognitive scores are rarely outside the normal range established in HCs. Specifically, no mTBI participant scored lower than HCs on TMT-A, TMT-B, EVMD, WMS, or IR. At most 39% of mTBI participants scored lower than HCs on EVMI (Table 2). Thus, on the one hand, mTBI participants in both samples have cognitive profiles representative of routinely observed mTBI syndromes. On the other hand, a clear majority of mTBI participants have cognitive profiles difficult to distinguish from those of HCs. This is not unlike many other reports of difficulties in distinguishing individual mTBI patients from HCs based solely on cognitive scores. In conclusion, the classifier was trained and validated on mTBI participants with cognitive profiles very similar to those of HCs. This suggests its applicability to persons with similarly quasi-normal cognition but whose mTBI diagnosis can benefit from its insights.

In may also be useful to compare this work to previous research to illustrate the significant advantages associated with this work. This effort was able to distinguish mTBI participants from HCs with accuracy above 99%. Of 25 supervised classifiers trained, the naïve Bayesian classifier demonstrates the highest classification accuracy and generalizability to an external independent validation sample whose acquisition and pre-processing pipeline differed from those of the discovery set. Of note, no other MRI-based classifier is known to have higher predictive accuracy. For example, in one instance, classifiers were trained to identify acute mTBI from resting-state functional MRIs (maximum accuracy: 84.1%) or diffusion MRIs (maximum accuracy: 75.5%). In one instance, a ML classifier was developed that used resting-state magnetoencephalography in patients with mTBI to achieve a classification accuracy of 79%. These classifiers, in contrast to that disclosed herein, failed to reach the high sensitivity and specificity thresholds expected in clinical settings.

Regarding interpretability, the classification ability of brain connections identified here highlight a small pool of connectivity alterations that may be uniquely useful for acute mTBI identification. Specifically, WM connections that are most useful to the classifier for identifying mTBI constitute a “canary in the coalmine” network of brain connectivity that is particularly sensitive to the neurological sequelae of head impacts.

Many connections idiosyncratic to mTBI involve the prefrontal cortex (PFC), which is essential to executive functioning and control of cognitive and motor abilities. Research highlights the PFC's role in the cognitive control of emotion and behaviour, where it exerts top-down influence on subcortical and limbic structures like the amygdala. Such disturbances frequently manifest themselves as anxiety or depression, whose severities are mediated by damage to fronto-limbic WM connections. Neurobehavioral disorders, socio-emotional dysregulation, and impulsivity can occur after mTBI due to fronto-subcortical and fronto-limbic disconnectivity. The prominence assigned by the classifier to connections between the PFC and both subcortical and limbic regions may help to explain the high incidence of affective disorders (23% of adults within 4 years of injury) and behavioural disturbances such as aggression (34% of adults within 6 months of injury) following mTBI. Many connections with near-ideal classification accuracy involve the superior frontal gyrus, the most voluminous gyrus in the parcellation. This long structure spans the superior extremity of dorsolateral PFC along the anteroposterior axis, where shear and strain forces are often strong during injury. Similarly, the connection between the superior frontal sulcus and the caudate nucleus spans a notable portion of frontal WM, where connectivity from superficial frontal areas travel to deeper subcortical regions. Thus, it is unsurprising to find these and similar structures among the set of connections with high classification accuracy.

Somatosensory and somatomotor areas are prominent in the “canary in the coal mine” network. Specifically, connectivity between inferior precentral and inferior temporal sulci is involved in nociceptive and somatomotor memory circuits, both of which are often affected by mTBI. These connections originate or terminate along the boundary between the somatomotor and somatosensory cortices; their high sensitivity to mTBI, as quantified here, may reflect the cortical dynamics of post-traumatic pain syndromes. Primary somatosensory cortex and several supplementary motor areas localize to the central sulcus and the superior parietal lobule, respectively. The significant traumatic disruption of connections involving these areas may explain the frequency of post-mTBI complaints pertaining to sensorimotor integration and balance problems.

Many connections salient to mTBI identified here belong to the ventral (‘what’) visual pathway subserving object recognition and storage of both visual and long-term memory. This pathway includes cortical structures such as the fusiform gyrus, the lateral occipito-temporal sulcus, and the inferior temporal sulcus. After injury, persons with mTBI exhibit impairments in object recognition through the ventral visual processing stream. Connectivity between (A) the superior frontal gyrus and both (B) parahippocampal gyrus and (C) lateral occipito-temporal sulcus also links attention to memory systems. Similarly, connections linking the hippocampus to the (ipsilateral) fusiform gyrus are involved in visual processing and memory. Both structures are vulnerable to mTBI partly due to their locations in the brain, which often sustain deformations and ensuing connectivity alterations during traumatic events. The disruption of connections between these structures may partly explain memory symptoms that are common after mTBI, as seen in some mTBI patients' lower-than-normal RAVLT, EVMI/EVMD and WMS scores.

Connectivity between the superior part of the precentral sulcus (located in the dorsolateral PFC) and the pericallosal sulcus (located in the limbic lobe) perfectly predicts mTBI status in the validation sample. WM tracts innervating the pericallosal sulcus exhibit altered connectivity properties in preclinical Alzheimer's disease. Because pericallosal and precentral areas mediate cognitive functions affected by both mTBI and Alzheimer's disease, the connectivity identified here may suggest neurocognitive parallels between these conditions that have been proposed elsewhere.

Reproducible classification with accuracy above 95% is difficult in medicine and life sciences and therefore warrants skepticism. To strengthen trust in the classifier, this effort compared mean FAs between groups across all bilateral pairs of connectivity features, including those that were either most or least salient to the classifier. Because FA is a measure of water diffusion measured by MRI, one may reason that this type of analysis could relate abstract features identified by the Bayesian classifier to concrete measures of brain connectivity. The results indicate, as expected, that connections salient to the classifier exhibit significant mean FA differences between diagnostic groups. Conversely, connections with poor saliency have nonsignificant group differences in mean FA. These results strengthen the premise that the classifier relies on physical alterations effected by mTBI on connectivity features rather than on spurious properties of WM streamline bundles.

In this study, 50 features (˜4%) have individual classification accuracies above 95%. Since 13 features were used for classification, this suggests that accurate binary classification of mTBI is possible based on connection pairs other than those used here, as long as such classification relies on this pool of highly salient features. If the pool size were (nearly) equal to the number of features used for classification, there would be concern that the discriminant ability of the classifier is sensitive to numerical instability pertaining to model architecture and related considerations. In the present case, however, the availability of a much larger pool of salient features suggests that the diagnostic strategy is robust to numerical analysis choices pertaining to optimization scheme, hyperparameters, etc. Importantly, overfitting is more likely when the salient feature pool is small relative to the number of classifier features. This is partly because a paucity of salient features suggests poor class separation in the eigenspace of the discriminant function. The fact that the salient feature pool is ˜4 times larger than the number of features used for classification alleviates concerns that the classifier relies on overfit features, or that our accuracy may be irreproducible or ungeneralizable.

Classifiers may be tested on MRIs acquired from both (A) acute mTBI patients without radiological findings and (B) matched HCs. For this reason, it may be challenging to identify cohorts suitable for such a study. Indeed, the validation sample comprises two independent subsamples from distinct studies where participants were imaged on the same type of scanner but using slightly different scan parameters. For this reason, in the validation sample, the accuracy of the classification is partly affected by the statistical interaction between diagnostic status and scan protocol. Specifically, in the USC validation subsample, the true positive (100%) and false negative (0%) rates were calculated. The false positive and true negative rate are unknown because there are no HCs in this subsample. Similarly, in the HCP validation subsample, the true negative rate is 100% and the false positive rate is 0%; the true positive and false negative rates are unknown because there are no mTBI participants in this subsample. One may expect this confound to have only a modest effect on classification accuracy for several reasons. Firstly, the classifier-despite being trained on the discovery sample-performed very well on the validation sample. If the training had identified mTBI features which transferred poorly to the validation sample (where these features were tested), the latter's classification performance rates would not have been ideal, especially if the validation subsamples were independent. However, the USC and HCP subsamples are independent, and the classifier's classification rates are excellent both in each distinct subsample as well as in the (combined) validation sample. These findings suggest that the classification features have both genuine discriminative power and potential generalizability. Secondly, the ability to demonstrate high ML classification ability despite potential confounds is desirable because it provides evidence of model generalizability. Nevertheless, data from other independent cohorts can further validate the approach and evince its generalizability to new samples. Such testing should also appraise how different neuroimage analysis methods for brain segmentation, tractography, connectomic analysis, etc. affect results. Finally, although classification accuracy is very high for both the discovery and validation samples, the trustworthiness of this metric is dependent on sample size and population variance. Thus, there may be value in replication in larger samples that capture this variance.

Turning now to a comparison to clinical and cognitive measures, the discussion continues. Routine methods of diagnosing mTBI rely on the ability to measure GCS scores, loss of consciousness, and post-traumatic amnesia within 30 minutes of the injury. However, due to typical delays in clinical assessment or to the delayed effects of many injuries, some researchers estimate that 50%-90% of mTBI cases are not identified early enough to reduce chance of sequelae. There are also physiological factors whose confounding effects may contribute to missed mTBI diagnoses. For example, mTBI symptoms are often misattributed to alcohol intoxication, whose relationship to GCS remains unclear. Because 35%-50% of suspected mTBI patients may be intoxicated at the time of injury, it is important to develop novel diagnostic protocols that are less vulnerable to intoxication status. Also, in patients with acute polytrauma, cognitive assessments often lead to missed mTBI diagnoses (60.9% of cases) if other injuries are accompanied by symptoms similar to those of mTBI.

The neuropsychological assessments used to ascertain the presence of sequelae indicating injury to the brain include the BTACT, RAVLT, TMT, and WAIS indices documented in this study, which are reportedly related to cognitive outcome. For example, whereas the RAVLT is often used to test verbal memory and learning in patients, one may posit that the RAVLT also assesses global cognitive functions in the medical rehabilitation setting. On average, in both the discovery and validation samples, acute mTBI participants had significantly poorer verbal memory compared to HCs, suggesting that tests such as the RAVLT could differentiate between these groups. However, in practice, the RAVLT has substantially lower accuracy (69%) than the classifier (>99%) when used in the context of mTBI diagnosis. Similarly, 93% of mTBI patients had normal RAVLT scores. The RAVLT also has low accuracy in predicting recovery for patients with moderate-to-severe TBI. The BTACT was designed to assess typical aging. Although it is often used to follow up on cognitive performance after mTBI, one may argue that this test has low criterion validity for mTBI at the acute stage. One may content that the classifier's best utility is for detecting TBI sequelae rather than differentiating between participants according to diagnostic status. One may observe significant deficits in BTACT performance, on average, in acute mTBI participants compared to MIDUS HCs. However, the vast majority of mTBI patients had BTACT scores within the normal range (ranging from 100% on EVMD, WMS, and IR, to 61% on EVMI, Table 2). Unsurprisingly, the BTACT was insufficient for sensitive and specific differentiation between mTBI and HC. These limitations of routine neuropsychological assessment used to diagnose mTBI highlight the need for a novel, robust and independent source of clinical and scientific evidence such as the one introduced here.

Various limitations may be relevant for discussion. For instance, in the validation sample, the average age of mTBI participants differs significantly from that of HCs (Table 1). This discrepancy was partially mitigated by regressing out age, sex, and their interactions from classifier features. In future studies, the classifier should be tested on additional independent mTBI cohorts where participants are matched on age, sex, and other demographic variables (e.g., years of education, socioeconomic status, and race) known to affect mTBI functional outcomes. Furthermore, classifier robustness to age- and sex-related effects should be studied. Classification accuracy should also be investigated in the presence of additional sources of diagnostic information such as the GCS, T₂-weighted MRI, CT, and measures of functional disability, cognition, and level of consciousness.

This disclosure introduces a novel approach for identifying mTBI in the absence of neuroradiological findings using ML of connectomic brain features. The classifier leverages “canary in the coalmine” connectivity features to achieve near-ideal classification. In the discovery sample, the protocol identifies acute mTBI with high sensitivity and specificity even in patients with quasi-normal cognitive scores. The need for methods like those herein increases as the incidence of mTBI rises within aging populations. These findings could also help to identify novel biomarkers that reflect more accurately how acute mTBI changes the connectome.

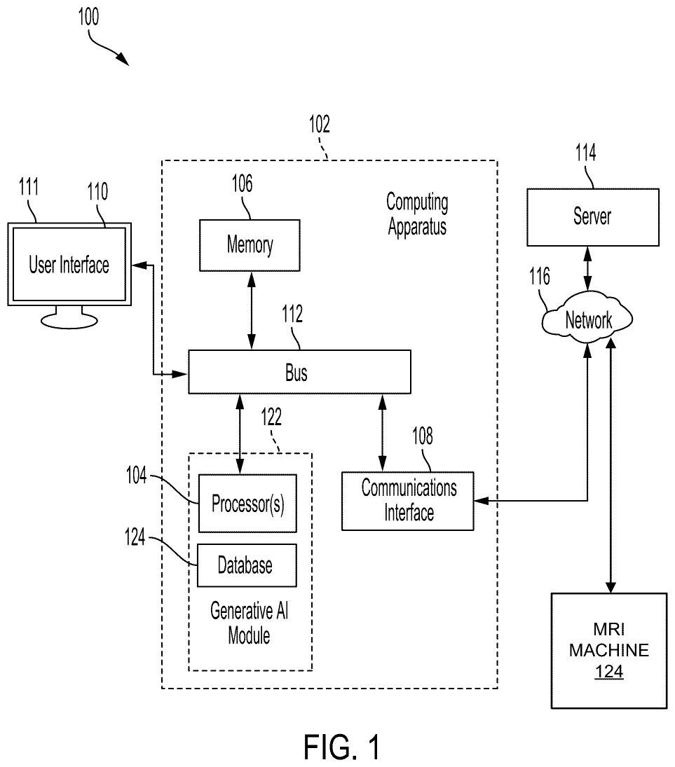

Referring now to FIG. 1, but with occasional reference to the features illustrated in FIGS. 2-8, a system 100 for accurate early diagnosis of mild traumatic brain injury (mTBI) to prevent sequelae and improve neurocognitive outcomes is disclosed herein. The system 100 (e.g., a computing system) may include a computing apparatus 102. The computing apparatus 102 may include one or more processors 104, one or more memories 106 and/or one or more buses 112 and/or other mechanisms for communicating between and among the one or more processors 104 and the one or more memories 106. The computing apparatus 102 can be a Bayesian machine learning classifier. The system 100 may be a cloud computing system including processors, servers, storage, databases, networking, software, analytics, and/or intelligence accessed or performed over or using the Internet (“the cloud”). The one or more processors 104 may be implemented as a single processor or as multiple processors. The one or more processors 104 may execute instructions stored in the memory 106 to implement the applications and/or detection of the system 100.

The one or more processors 104 may be coupled to the memory 106. The memory 106 may include one or more of a Random Access Memory (RAM) or other volatile or non-volatile memory. The memory 106 may be a non-transitory memory or a data storage device, such as a hard disk drive, a solid-state disk drive, a hybrid disk drive, or other appropriate data storage, and may further store machine-readable instructions, which may be loaded and executed by the one or more processors 104.

The memory 106 may include one or more of random-access memory (“RAM”), static memory, cache, flash memory and any other suitable type of storage device or computer readable storage medium, which is used for storing instructions to be executed by the one or more processors 104. The storage device or the computer readable storage medium may be a read only memory (“ROM”), flash memory, and/or memory card, that may be coupled to a bus 112 or other communication mechanism. The storage device may be a mass storage device, such as a magnetic disk, optical disk, and/or flash disk that may be directly or indirectly, temporarily, or semi-permanently coupled to the bus 112 or other communication mechanism and be electrically coupled to some or all the other components within the system 100 including the memory 106, the user interface 110 and/or the communications interface 108 via the bus 112.

The term “computer-readable medium” is used to define any medium that can store and provide instructions and other data to a processor, particularly where the instructions are to be executed by a processor and/or other peripheral of the processing system. Such medium can include non-volatile storage, volatile storage, and transmission media. Non-volatile storage may be embodied on media such as optical or magnetic disks. Storage may be provided locally and in physical proximity to a processor or remotely, typically by use of network connection. Non-volatile storage may be removable from computing system, as in storage or memory cards or sticks that can be easily connected or disconnected from a computer using a standard interface.

The system 100 may include a user interface 110. The user interface 110 may include an input/output device. The input/output device may receive user input, such as a user interface element, hand-held controller that provides tactile/proprioceptive feedback, a button, a dial, a microphone, a keyboard, or a touch screen, and/or provides output, such as a display, a speaker, an audio and/or visual indicator, or a refreshable braille display. The display may be a computer display, a tablet display, a mobile phone display, an augmented reality display or a virtual reality headset. The display may output or provide accurate early diagnosis results of mild traumatic brain injury (mTBI).

The user interface 110 may include an input/output device that receives user input, such as a user interface element, a button, a dial, a microphone, a keyboard, or a touch screen, and/or provides output, such as a display, a speaker, headphones, an audio and/or visual indicator, a device that provides tactile/proprioceptive feedback or a refreshable braille display. The speaker may be used to output audio associated with the audio conference and/or the video conference. The user interface 110 may receive user input that may include configuration settings for one or more user preferences, such as a selection of joining an audio conference or a video conference when both options are available, for example.

The system 100 may have a network 116 connected to a server 114. The network 116 may be a local area network (LAN), a wide area network (WAN), a cellular network, the Internet, or combination thereof, that connects, couples and/or otherwise communicates between the various components of the system 100 with the server 114. The server 114 may be a remote computing device or system that includes a memory, a processor and/or a network access device coupled together via a bus. The server 114 may be a computer in a network that is used to provide services, such as accessing files or sharing peripherals, to other computers in the network.

The system 100 may include a communications interface 108, such as a network access device. The communications interface 108 may include a communication port or channel, such as one or more of a Dedicated Short-Range Communication (DSRC) unit, a Wi-Fi unit, a Bluetooth® unit, a radio frequency identification (RFID) tag or reader, or a cellular network unit for accessing a cellular network (such as 3G, 4G or 5G). The communication interface may transmit data to and receive data from the different components.

The server 114 may include a database. A database is any collection of pieces of information that is organized for search and retrieval, such as by a computer, and the database may be organized in tables, schemas, queries, reports, or any other data structures. A database may use any number of database management systems. The information may include real-time information, periodically updated information, or user-inputted information.

In various embodiments, the computing apparatus 102 can include a generative artificial intelligence (“AI”) module 122. The generative AI module 122 can include the one or more processors 104. Stated another way, the generative AI module 122 can be run, or operated by the one or more processors 104. In various embodiments, the generative AI module 122 can perform the steps of the methods claimed herein and output or provide cross-session normalization of brain recordings using sensory tasks alongside a primary task of interest to the user interface 110. The generative AI module 112 may be a Bayesian machine learning classier.

In various embodiments, the generative AI module 122 is configured to decode cross-session normalization of brain recordings from a database 124 as described further herein. In this regard, the generative AI module 122 is configured to update a generative AI model based on the brain signals and data received from the database 124. In various embodiments, the brain signals from the generative AI module 122, as described further herein can include the methods described herein.

The system 100 may perform various functions. For instance, the system 100 for accurate early diagnosis for mild traumatic brain injury (mTBI) may perform methods to prevent sequelae and improve neurocognitive outcomes. For instance, the system 100 may include an imaging machine. The system 100 may be connected to an imaging machine, directly or via the network 116. The imaging machine may be an MRI machine 124. In other instances, the so-called imaging machine is a memory 106 that has images collected by an imaging machine, such as an MRI machine or other various imaging data.

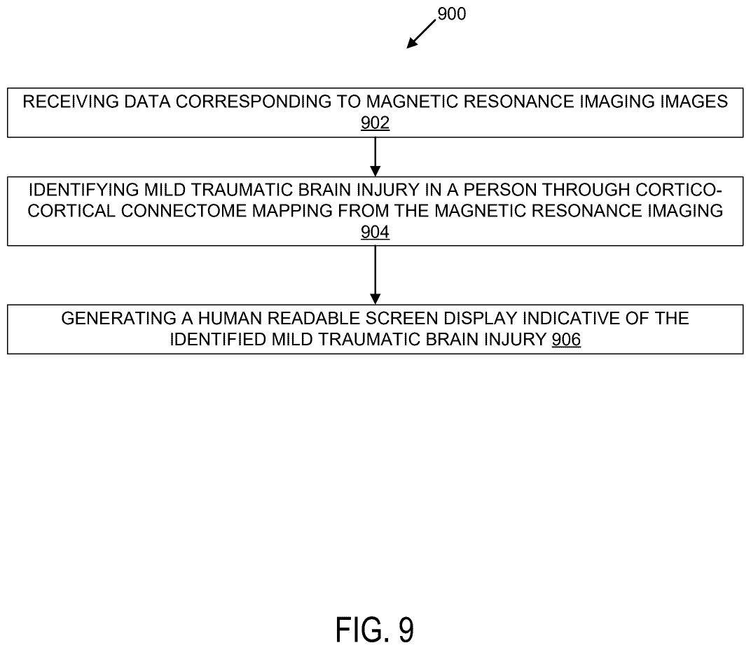

Turning now to both FIG. 1 and FIG. 9, the system 100 may implement a method 900 including various aspects. For instance, the method 900 may be a method for diagnosis of mild traumatic brain injury (mTBI) to prevent sequelae and improve neurocognitive outcomes. The method may include receiving data corresponding to magnetic resonance imaging images (block 902). The method may include identifying, using a Bayesian machine learning classifier, mild traumatic brain injury in a person through cortico-cortical connectome mapping from the magnetic resonance imaging (block 904). The method may include generating a human readable screen display indicative of the identified mild traumatic brain injury (block 906).

In various instances, the Bayesian machine learning classifier is a processor 104 and/or software algorithm running on the processor 104, configured to identify acute mTBI in the absence of neuroradiological MRI findings on T1- or T2-weighted scans. In various instances, the Bayesian machine learning classifier is a processor 104 and/or software algorithm running on the processor 104, configured to discriminate or reveal TAI-related white matter (WM) connections or descriptors that are unusually sensitive to mTBI. In various instances, the Bayesian machine learning classifier is a processor 104 and/or software algorithm running on the processor 104, configured to perform a bilateral saliency analysis to alleviate the potential confounds of asymmetrical mTBI effects on neurocircuitry.

In various instances, the cortico-cortical connectome mapping includes analyzing multiple bilateral cortico-cortical connection pairs relating to (e.g., from or forming) classification features. The bilateral cortico-cortical connection pairs may link frontal lobes to limbic, temporal, parietal, and occipital structures. The bilateral cortico-cortical connection pairs may include 13 bilateral cortico-cortical connection pairs. The cortical structures linked by the 13 connections may be displayed on the cortex, such as a computer screen display having an image representing the cortex. In various instances, one connection links the frontal lobe to subcortical structures and to the parietal lobe, both ipsilaterally and contralaterally. In various instances, one connection between the superior frontal sulcus and the caudate nucleus spans a notable portion of frontal WM, where connectivity from superficial frontal areas travel to deeper subcortical regions. In various instances, these connections originate or terminate along the boundary between the somatomotor and somatosensory cortices, and their high sensitivity to mTBI reflects the cortical dynamics of post-traumatic pain syndromes. In various instances, the connectivity between the superior part of the precentral sulcus (located in the dorsolateral PFC) and the pericallosal sulcus (located in the limbic lobe) predicts and displays mTBI status on a display screen.

The system 100 may perform various functions. For instance, the system 100 for accurate early diagnosis of mild traumatic brain injury (mTBI) may perform methods to prevent sequelae and improve neurocognitive outcomes. For instance, the system 100 may receive from an imaging machine, such as an MRI machine, or from a memory that has images collected by an imaging machine, such as an MRI machine, various imaging data.

The system may have a computing apparatus 102 which may be a Bayesian machine learning classifier, and/or may include one or more processors 104, memories 106, and the like as described.

In various instances, the Bayesian machine learning classifier is a processor 104 and/or software algorithm running on the processor 104, configured to identify acute mTBI in the absence of neuroradiological MRI findings on T1- or T2-weighted scans. In various instances, the Bayesian machine learning classifier is a processor 104 and/or software algorithm running on the processor 104, configured to discriminate or reveal TAI-related white matter (WM) connections or descriptors that are unusually sensitive to mTBI. In various instances, the Bayesian machine learning classifier is a processor 104 and/or software algorithm running on the processor 104, configured to perform a bilateral saliency analysis to alleviate the potential confounds of asymmetrical mTBI effects on neurocircuitry.

In various instances, the cortico-cortical connectome mapping includes analyzing multiple bilateral cortico-cortical connection pairs that are related to (e.g., from or forming) classification features. The bilateral cortico-cortical connection pairs may link frontal lobes to limbic, temporal, parietal, and occipital structures. The bilateral cortico-cortical connection pairs may include 13 bilateral cortico-cortical connection pairs. The cortical structures linked by the 13 connections may be displayed on the cortex, such as a computer screen display having an image representing the cortex. In various instances, one connection links the frontal lobe to subcortical structures and to the parietal lobe, both ipsilaterally and contralaterally. In various instances, one connection between the superior frontal sulcus and the caudate nucleus spans a notable portion of frontal WM, where connectivity from superficial frontal areas travel to deeper subcortical regions. In various instances, these connections originate or terminate along the boundary between the somatomotor and somatosensory cortices, and their high sensitivity to mTBI reflects the cortical dynamics of post-traumatic pain syndromes. In various instances, the connectivity between the superior part of the precentral sulcus (located in the dorsolateral PFC) and the pericallosal sulcus (located in the limbic lobe) predicts and displays mTBI status on a display screen.

The detailed description of various embodiments herein makes reference to the accompanying drawings, which show various embodiments by way of illustration. While these various embodiments are described in sufficient detail to enable those skilled in the art to practice the disclosure, it should be understood that other embodiments may be realized and that logical chemical, electrical, and mechanical changes may be made without departing from the spirit and scope of the disclosure. Thus, the detailed description herein is presented for purposes of illustration only and not of limitation.

For example, the steps recited in any of the method or process descriptions may be executed in any suitable order and are not necessarily limited to the order presented. Furthermore, any reference to singular includes plural embodiments, and any reference to more than one component or step may include a singular embodiment or step. Also, any reference to attached, fixed, connected, or the like may include permanent, removable, temporary, partial, full, and/or any other possible attachment option. Additionally, any reference to without contact (or similar phrases) may also include reduced contact or minimal contact.

The detailed description of various embodiments herein makes reference to the accompanying drawings and pictures, which show various embodiments by way of illustration. While these various embodiments are described in sufficient detail to enable those skilled in the art to practice the disclosure, it should be understood that other embodiments may be realized, and that logical and mechanical changes may be made without departing from the spirit and scope of the disclosure. Thus, the detailed description herein is presented for purposes of illustration only and not for purposes of limitation.

For example, the steps recited in any of the method or process descriptions may be executed in any order and are not limited to the order presented. Moreover, any of the functions or steps may be outsourced to or performed by one or more third parties. Modifications, additions, or omissions may be made to the systems, apparatuses, and methods described herein without departing from the scope of the disclosure. For example, the components of the systems and apparatuses may be integrated or separated. An individual component may be comprised of two or more smaller components that may provide a similar functionality as the individual component. Moreover, the operations of the systems and apparatuses disclosed herein may be performed by more, fewer, or other components and the methods described may include more, fewer, or other steps. Additionally, steps may be performed in any suitable order. As used in this document, “each” refers to each member of a set or each member of a subset of a set. Furthermore, any reference to singular includes plural embodiments, and any reference to more than one component may include a singular embodiment. Use of ‘a’ or ‘an’ before a noun naming an object shall indicate that the phrase be construed to mean ‘one or more’ unless the context sufficiently indicates otherwise. For example, the description or claims may refer to a processor for convenience, but the invention and claim scope contemplates that the processor may be multiple processors. The multiple processors may handle separate tasks or combine to handle certain tasks. Although specific advantages have been enumerated herein, various embodiments may include some, none, or all of the enumerated advantages. A “processor” may include hardware that runs the computer program code. Specifically, the term ‘processor’ may be synonymous with terms like controller and computer and should be understood to encompass not only computers having different architectures such as single/multi-processor architectures and sequential (Von Neumann)/parallel architectures but also specialized circuits such as field-programmable gate arrays (FPGA), application specific circuits (ASIC), signal processing devices and other devices.

Systems, methods, and computer program products are provided. In the detailed description herein, references to “various embodiments,” “one embodiment,” “an embodiment,” “an example embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described. After reading the description, it will be apparent to one skilled in the relevant art(s) how to implement the disclosure in alternative embodiments.

The system may allow users and/or electronic devices (collectively, “users”) to access data, and receive updated data in real time from other users. The system may store the data (e.g., in a standardized format) in a plurality of storage devices, provide remote access over a network so that users may update the data in a non-standardized format (e.g., dependent on the hardware and software platform used by the user) in real time through a GUI, convert the updated data that was input (e.g., by a user) in a non-standardized form to the standardized format, automatically generate a message (e.g., containing the updated data) whenever the updated data is stored and transmit the message to the users over a computer network in real time, so that the user has immediate access to the up-to-date data. The system allows remote users to share data in real time in a standardized format, regardless of the format (e.g. non-standardized) that the information was input by the user. The system may also include a filtering tool that is remote from the end user and provides customizable filtering features to each end user. The filtering tool may provide customizable filtering by filtering access to the data. The filtering tool may identify data or accounts that communicate with the server and may associate a request for content with the individual account, user, device, etc. The system may include a filter on a local computer and a filter on a server.

The terms “first,” “second,” “third,” “fourth,” and the like in the description and in the claims, if any, are used for distinguishing between similar elements and not necessarily for describing a particular sequential or chronological order. It is to be understood that the terms so used are interchangeable under appropriate circumstances such that the embodiments described herein are, for example, capable of operation in sequences other than those illustrated or otherwise described herein. Furthermore, the terms “include,” and “have,” and any variations thereof, are intended to cover a non-exclusive inclusion, such that a process, method, system, article, device, or apparatus that comprises a list of elements is not necessarily limited to those elements but may include other elements not expressly listed or inherent to such process, method, system, article, device, or apparatus.

The terms “left,” “right,” “front,” “back,” “top,” “bottom,” “over,” “under,” and the like in the description and in the claims, if any, are used for descriptive purposes and not necessarily for describing permanent relative positions. It is to be understood that the terms so used are interchangeable under appropriate circumstances such that the embodiments of the apparatus, methods, and/or articles of manufacture described herein are, for example, capable of operation in other orientations than those illustrated or otherwise described herein.

The terms “couple,” “coupled,” “couples,” “coupling,” and the like should be broadly understood and refer to connecting two or more elements mechanically and/or otherwise. Two or more electrical elements may be electrically coupled together, but not be mechanically or otherwise coupled together. Coupling may be for any length of time, e.g., permanent or semi-permanent or only for an instant. “Electrical coupling” and the like should be broadly understood and include electrical coupling of all types. The absence of the word “removably,” “removable,” and the like near the word “coupled,” and the like does not mean that the coupling, etc. in question is or is not removable.

As defined herein, two or more elements are “integral” if they are comprised of the same piece of material. As defined herein, two or more elements are “non-integral” if each is comprised of a different piece of material.

As defined herein, “real-time” can, in some embodiments, be defined with respect to operations carried out as soon as practically possible upon occurrence of a triggering event. A triggering event can include receipt of data necessary to execute a task or to otherwise process information. Because of delays inherent in transmission and/or in computing speeds, the term “real time” encompasses operations that occur in “near” real time or somewhat delayed from a triggering event. In a number of embodiments, “real time” can mean real time less a time delay for processing (e.g., determining) and/or transmitting data. The particular time delay can vary depending on the type and/or amount of the data, the processing speeds of the hardware, the transmission capability of the communication hardware, the transmission distance, etc. However, in many embodiments, the time delay can be less than approximately one second, two seconds, five seconds, or ten seconds.

As defined herein, “approximately” can, in some embodiments, mean within plus or minus ten percent of the stated value. In other embodiments, “approximately” can mean within plus or minus five percent of the stated value. In further embodiments, “approximately” can mean within plus or minus three percent of the stated value. In yet other embodiments, “approximately” can mean within plus or minus one percent of the stated value.

As used herein, “satisfy,” “meet,” “match,” “associated with,” or similar phrases may include an identical match, a partial match, meeting certain criteria, matching a subset of data, a correlation, satisfying certain criteria, a correspondence, an association, an algorithmic relationship, and/or the like. Similarly, as used herein, “authenticate” or similar terms may include an exact authentication, a partial authentication, authenticating a subset of data, a correspondence, satisfying certain criteria, an association, an algorithmic relationship, and/or the like.

Terms and phrases similar to “associate” and/or “associating” may include tagging, flagging, correlating, using a look-up table or any other method or system for indicating or creating a relationship between elements. Moreover, the associating may occur at any point, in response to any suitable action, event, or period of time. The associating may occur at pre-determined intervals, periodically, randomly, once, more than once, or in response to a suitable request or action. Any of the information may be distributed and/or accessed via a software enabled link, wherein the link may be sent via an email, text, post, social network input, and/or any other method.

As used herein, “electronic communication” means communication of electronic signals with physical coupling (e.g., “electrical communication” or “electrically coupled”) or without physical coupling and via an electromagnetic field (e.g., “inductive communication” or “inductively coupled” or “inductive coupling”) and/or a radio frequency (RF) communications protocol. In this regard, “electronic communication,” as used herein, includes wired and wireless communications (e.g., Bluetooth, Bluetooth LE, NFC, TCP/IP, Wi-Fi, etc.).

Any databases discussed herein may include relational, hierarchical, graphical, blockchain, object-oriented structure, and/or any other database configurations. Any database may also include a flat file structure wherein data may be stored in a single file in the form of rows and columns, with no structure for indexing and no structural relationships between records. For example, a flat file structure may include a delimited text file, a CSV (comma-separated values) file, and/or any other suitable flat file structure. Common database products that may be used to implement the databases include DB2® by IBM® (Armonk, NY), various database products available from ORACLE® Corporation (Redwood Shores, CA), MICROSOFT ACCESS® or MICROSOFT SQL SERVER® by MICROSOFT® Corporation (Redmond, Washington), MYSQL® by MySQL AB (Uppsala, Sweden), MONGODB®, Redis, Apache Cassandra®, HBASE® by APACHE®, MapR-DB by the MAPR® corporation, or any other suitable database product. Moreover, any database may be organized in any suitable manner, for example, as data tables or lookup tables. Each record may be a single file, a series of files, a linked series of data fields, or any other data structure.

As used herein, data may refer to partially or fully structured, semi-structured, or unstructured data sets including “big data,” which may include millions of rows and hundreds of thousands or millions of columns.

Association of certain data may be accomplished through any desired data association technique such as those known or practiced in the art. For example, the association may be accomplished either manually or automatically. Automatic association techniques may include, for example, a database search, a database merge, GREP, AGREP, SQL, using a key field in the tables to speed searches, sequential searches through all the tables and files, sorting records in the file according to a known order to simplify lookup, and/or the like. The association step may be accomplished by a database merge function, for example, using a “key field” in pre-selected databases or data sectors. Various database tuning steps are contemplated to optimize database performance. For example, frequently used files such as indexes may be placed on separate file systems to reduce In/Out (“I/O”) bottlenecks.

One skilled in the art will also appreciate that, for security reasons, any databases, systems, devices, servers, or other components of the system may comprise or consist of any combination thereof at a single location or at multiple locations, wherein each database or system includes any of various suitable security features, such as firewalls, access codes, encryption, decryption, public and private keys, and/or the like.

As used herein, a “script” refers to instructions for a computing device to carry out one or more tasks automatically. As used herein, the term “network” includes any cloud, cloud computing system, or electronic communications system or method which incorporates hardware and/or software components. Communication among the parties may be accomplished through any suitable communication channels, such as, for example, a telephone network, an extranet, an intranet, internet, personal internet device, online communications, satellite communications, off-line communications, wireless communications, transponder communications, local area network (LAN), wide area network (WAN), virtual private network (VPN), networked or linked devices, keyboard, mouse, and/or any suitable communication or data input modality. Moreover, although the system may be described herein as being implemented with TCP/IP communications protocols, the system may also be implemented using IPX, APPLETALK®, IPv6, NetBIOS, any tunneling protocol (e.g. IPsec, SSH, etc.), or any number of existing or future protocols. If the network is in the nature of a public network, such as the internet, it may be advantageous to presume the network to be insecure and open to eavesdroppers. Specific information related to the protocols, standards, and application software utilized in connection with the internet is generally known to those skilled in the art and, as such, need not be detailed herein.

“Cloud” or “Cloud computing” or “cloud computing infrastructure” includes a model for enabling convenient, on-demand network access to a shared pool of configurable computing resources (e.g., networks, servers, storage, applications, and services) that can be rapidly provisioned and released with minimal management effort or service provider interaction. Cloud computing may include location-independent computing, whereby shared servers provide resources, software, and data to computers and other devices on demand. Reference to a “device” or processor or memory or the like may include cloud resources, non-cloud resources, or combinations of cloud and non-cloud resources.

Computer programs (also referred to as computer control logic) are stored in main memory and/or secondary memory. Computer programs may also be received via communications interface. These computer program instructions may be loaded onto a general purpose computer, special purpose computer, controller, or other programmable data processing apparatus to produce a machine, such that the instructions that execute on the computer or other programmable data processing apparatus create means for implementing the functions specified in the flowchart block or blocks. These computer program instructions may also be stored in a computer-readable memory that can direct a computer, controller, or other programmable data processing apparatus to function in a particular manner, such that the instructions stored in the computer-readable memory produce an article of manufacture including instruction means which implement the function specified in the flowchart block or blocks. The computer program instructions may also be loaded onto a computer or other programmable data processing apparatus to cause a series of operational steps to be performed on the computer or other programmable apparatus to produce a computer-implemented process such that the instructions which execute on the computer or other programmable apparatus provide steps for implementing the functions specified in the flowchart block or blocks.

In various embodiments, software may be stored in a computer program product and loaded into a computer system using a removable storage drive, hard disk drive, or communications interface. The control logic (software), when executed by the processor or controller, causes the processor or controller to perform the functions of various embodiments as described herein. In various embodiments, hardware components may take the form of application specific integrated circuits (ASICs). Implementation of the hardware so as to perform the functions described herein will be apparent to persons skilled in the relevant art(s).

As will be appreciated by one of ordinary skill in the art, the system may be embodied as a customization of an existing system, an add-on product, a processing apparatus executing upgraded software, a stand-alone system, a distributed system, a method, a data processing system, a device for data processing, and/or a computer program product. Accordingly, any portion of the system or a module may take the form of a processing apparatus executing code, an internet based embodiment (e.g., an internet-based driving command system), an entirely hardware embodiment, or an embodiment combining aspects of the internet, software, and hardware. Furthermore, the system may take the form of a computer program product on a computer-readable storage medium having computer-readable program code means embodied in the storage medium. Any suitable computer-readable storage medium may be utilized, including hard disks, solid state storage media, CD-ROM, BLU-RAY DISC®, optical storage devices, magnetic storage devices, and/or the like.

The system and method may be described herein in terms of functional block components, screen shots, optional selections, and various processing steps. It should be appreciated that such functional blocks may be realized by any number of hardware and/or software components configured to perform the specified functions. For example, the system may employ various integrated circuit components, e.g., memory elements, processing elements, logic elements, look-up tables, and the like, which may carry out a variety of functions under the control of one or more microprocessors or other control devices. Similarly, the software elements of the system may be implemented with any programming or scripting language such as C, C++, C#, JAVA®, JAVASCRIPT®, JAVASCRIPT® Object Notation (JSON), VBScript, Macromedia COLD FUSION, COBOL, MICROSOFT® company's Active Server Pages, assembly, PERL®, PHP, awk, PYTHON®, Visual Basic, SQL Stored Procedures, PL/SQL, any UNIX® shell script, and extensible markup language (XML) with the various algorithms being implemented with any combination of data structures, objects, processes, routines or other programming elements. Further, it should be noted that the system may employ any number of techniques for data transmission, signaling, data processing, network control, and the like. Still further, the system could be used to detect or prevent security issues with a client-side scripting language, such as JAVASCRIPT®, VBScript, or the like.

The system and method are described herein with reference to screen shots, block diagrams and flowchart illustrations of methods, apparatus, and computer program products according to various embodiments. It will be understood that each functional block of the block diagrams and the flowchart illustrations, and combinations of functional blocks in the block diagrams and flowchart illustrations, respectively, can be implemented by computer program instructions.

In various embodiments, components, modules, and/or engines of the systems may be implemented as applications or apps. Apps are typically deployed in the context of a mobile operating system, including for example, a WINDOWS® mobile operating system, an ANDROID® operating system, an APPLE® iOS operating system, a BLACKBERRY® company's operating system, and the like. The app may be configured to leverage the resources of the larger operating system and associated hardware via a set of predetermined rules which govern the operations of various operating systems and hardware resources. For example, where an app desires to communicate with a device or network other than the mobile device or mobile operating system, the app may leverage the communication protocol of the operating system and associated device hardware under the predetermined rules of the mobile operating system. Moreover, where the app desires an input from a user, the app may be configured to request a response from the operating system which monitors various hardware components and then communicates a detected input from the hardware to the app.

Accordingly, functional blocks of the block diagrams and flowchart illustrations support combinations of means for performing the specified functions, combinations of steps for performing the specified functions, and program instruction means for performing the specified functions. It will also be understood that each functional block of the block diagrams and flowchart illustrations, and combinations of functional blocks in the block diagrams and flowchart illustrations, can be implemented by either special purpose hardware-based computer systems which perform the specified functions or steps, or suitable combinations of special purpose hardware and computer instructions. Further, illustrations of the process flows, and the descriptions thereof may make reference to user WINDOWS®/LINUX®/UNIX® applications, webpages, websites, web forms, prompts, etc. Practitioners will appreciate that the illustrated steps described herein may comprise, in any number of configurations, including the use of WINDOWS®/LINUX®/UNIX® applications, webpages, web forms, popup WINDOWS®/LINUX®/UNIX® applications, prompts, and the like. It should be further appreciated that the multiple steps as illustrated and described may be combined into single webpages and/or WINDOWS®/LINUX®/UNIX® applications but have been expanded for the sake of simplicity. In other cases, steps illustrated and described as single process steps may be separated into multiple webpages and/or WINDOWS®/LINUX®/UNIX® applications but have been combined for simplicity.

The computers discussed herein may provide a suitable website or other internet-based graphical user interface (GUI) which is accessible by users. In one embodiment, MICROSOFT® company's Internet Information Services (IIS), Transaction Server (MTS) service, and an SQL SERVER® database, are used in conjunction with MICROSOFT® operating systems, WINDOWS NT® web server software, SQL SERVER® database, and MICROSOFT® Commerce Server. Additionally, components such as ACCESS® software, SQL SERVER® database, ORACLE® software, SYBASE® software, INFORMIX® software, MYSQL® software, INTERBASE® software, etc., may be used to provide an Active Data Object (ADO) compliant database management system. In one embodiment, the APACHE® web server is used in conjunction with a LINUX® operating system, a MYSQL® database, and PHP, Ruby, and/or PYTHON® programming languages.

The term “non-transitory” is to be understood to remove only propagating transitory signals per se from the claim scope and does not relinquish rights to all standard computer-readable media that are not only propagating transitory signals per se. Stated another way, the meaning of the term “non-transitory computer-readable medium” and “non-transitory computer-readable storage medium” should be construed to exclude only those types of transitory computer-readable media which were found in In re Nuijten to fall outside the scope of patentable subject matter under 35 U.S.C. § 101.

Benefits, other advantages, and solutions to problems have been described herein with regard to specific embodiments. Furthermore, the connecting lines shown in the various figures contained herein are intended to represent exemplary functional relationships and/or physical couplings between the various elements. It should be noted that many alternative or additional functional relationships or physical connections may be present in a practical system. However, the benefits, advantages, solutions to problems, and any elements that may cause any benefit, advantage, or solution to occur or become more pronounced are not to be construed as critical, required, or essential features or elements of the disclosure. The scope of the disclosure is accordingly to be limited by nothing other than the appended claims, in which reference to an element in the singular is not intended to mean “one and only one” unless explicitly so stated, but rather “one or more.” Moreover, where a phrase similar to “at least one of A, B, or C” is used in the claims, it is intended that the phrase be interpreted to mean that A alone may be present in an embodiment, B alone may be present in an embodiment, C alone may be present in an embodiment, or that any combination of the elements A, B and C may be present in a single embodiment; for example, A and B, A and C, B and C, or A and B and C. Different cross-hatching may be used throughout the figures to denote different parts but not necessarily to denote the same or different materials.

Methods, systems, and articles are provided herein. In the detailed description herein, references to “one embodiment,” “an embodiment,” “various embodiments,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described. After reading the description, it will be apparent to one skilled in the relevant art(s) how to implement the disclosure in alternative embodiments.

Furthermore, no element, component, or method step in the present disclosure is intended to be dedicated to the public regardless of whether the element, component, or method step is explicitly recited in the claims. No claim element herein is to be construed under the provisions of 35 U.S.C. 112(f) unless the element is expressly recited using the phrase “means for.” As used herein, the terms “comprises,” “comprising,” or any other variation thereof, are intended to cover a non-exclusive inclusion, such that a process, method, article, or apparatus that comprises a list of elements does not include only those elements but may include other elements not expressly listed or inherent to such process, method, article, or apparatus.

1. A system for diagnosis of mild traumatic brain injury (mTBI) to prevent sequelae and improve neurocognitive outcomes, the system comprising:
an imaging device comprising a magnetic resonance imaging (MRI) machine configured to collect magnetic resonance images;
a processor connected to the imaging device to receive the magnetic resonance images and comprising a Bayesian machine learning classifier,
wherein the processor identifies, using the Bayesian machine learning classifier, mild traumatic brain injury in a person through cortico-cortical connectome mapping from the magnetic resonance images; and
a screen display connected to the processor and configured to output a human readable depiction of the identifying.

2. The system of claim 1, wherein the Bayesian machine learning classifier is a software algorithm running on the processor, configured to identify acute mTBI in the absence of neuroradiological MRI findings on T1- or T2-weighted scans.

3. The system of claim 1, wherein the Bayesian machine learning classifier is a software algorithm running on the processor, configured to discriminate or reveal TAI-related white matter (WM) connections or descriptors that are unusually sensitive to mTBI.

4. The system of claim 1, wherein the Bayesian machine learning classifier is a software algorithm running on the processor, configured to perform a bilateral saliency analysis to alleviate the potential confounds of asymmetrical mTBI effects on neurocircuitry.

5. The system of claim 1, wherein the cortico-cortical connectome mapping includes analyzing, by the processor, multiple bilateral cortico-cortical connection pairs from classification features.

6. The system of claim 5, wherein the bilateral cortico-cortical connection pairs link frontal lobes to limbic, temporal, parietal, and occipital structures, wherein the bilateral cortico-cortical connection pairs include 13 bilateral cortico-cortical connection pairs.

7. The system of claim 6, wherein the cortical structures linked by the 13 connections are displayed on a visual illustration of the cortex shown on the screen display.

8. The system of claim 6, wherein the connectivity between the superior part of the precentral sulcus (located in the dorsolateral PFC) and the pericallosal sulcus (located in the limbic lobe) predicts and displays mTBI status on a display screen.

9. A method for diagnosis of mild traumatic brain injury (mTBI) to prevent sequelae and improve neurocognitive outcomes, the method comprising:
receiving by a processor and from a magnetic resonance imaging machine data corresponding to magnetic resonance imaging images;
identifying, using a Bayesian machine learning classifier of the processor, mild traumatic brain injury in a person through cortico-cortical connectome mapping from magnetic resonance imaging; and
generating, by the processor, a human readable screen display indicative of the identified mild traumatic brain injury.

10. The method of claim 9, wherein the Bayesian machine learning classifier is a processor and/or software algorithm running on the processor, configured to identify acute mTBI in the absence of neuroradiological MRI findings on T₁- or T₂-weighted scans.

11. The method of claim 9, wherein the Bayesian machine learning classifier is a processor and/or software algorithm running on the processor, configured to discriminate or reveal TAI-related white matter (WM) connections or descriptors that are unusually sensitive to mTBI.

12. The method of claim 9, wherein the Bayesian machine learning classifier is a processor and/or software algorithm running on the processor, configured to perform a bilateral saliency analysis to alleviate the potential confounds of asymmetrical mTBI effects on neurocircuitry.

13. The method of claim 9, wherein the cortico-cortical connectome mapping includes analyzing multiple bilateral cortico-cortical connection pairs from classification features.

14. The method of claim 13, wherein the bilateral cortico-cortical connection pairs link frontal lobes to limbic, temporal, parietal, and occipital structures.

15. The method of claim 14, wherein the bilateral cortico-cortical connection pairs include 13 bilateral cortico-cortical connection pairs.

16. The method of claim 15, wherein the cortical structures linked by the 13 connections are displayed on the cortex.

17. The method of claim 16, wherein one connection links the frontal lobe to subcortical structures and to the parietal lobe, both ipsilaterally and contralaterally.

18. The method of claim 15, wherein one connection between the superior frontal sulcus and the caudate nucleus spans a notable portion of frontal WM, where connectivity from superficial frontal areas travel to deeper subcortical regions.

19. The method of claim 15, wherein these connections originate or terminate along the boundary between the somatomotor and somatosensory cortices, and their high sensitivity to mTBI reflects the cortical dynamics of post-traumatic pain syndromes.

20. The method of claim 15, wherein the connectivity between the superior part of the precentral sulcus (located in the dorsolateral PFC) and the pericallosal sulcus (located in the limbic lobe) predicts and displays mTBI status on a display screen.
